(19) United States
(12) Patent Application Publication
(10) Pub. No.: US 2013/0230899 A1
(43) Pub. Date: Sep. 5, 2013

(54) THERAPEUTIC AGENT FOR ARTERIOSCLEROSIS OR ARTERIOSCLEROTIC DISEASE, AND DIAGNOSTIC AGENT FOR ARTERIOSCLEROSIS OR ARTERIOSCLEROTIC DISEASE

(71) Applicants: Chuwa Tei, Kagoshima-shi (JP); Masaaki Miyata, Kagoshima-shi (JP); Yuko Furusyo, Kagoshima-shi (JP); Takami Matsuyama, Kagoshima-shi (JP); Taku Nagai, Kagoshima-shi (JP)

(72) Inventors: Chuwa Tei, Kagoshima-shi (JP); Masaaki Miyata, Kagoshima-shi (JP); Yuko Furusyo, Kagoshima-shi (JP); Takami Matsuyama, Kagoshima-shi (JP); Taku Nagai, Kagoshima-shi (JP)

(73) Assignees: Masaaki Miyata, Kagoshima-shi, Kagoshima (JP); Taku Nagai, Kagoshima-shi, Kagoshima (JP); Takami Matsuyama, Kagoshima-shi, Kagoshima (JP)

(21) Appl. No.: 13/884,215

(22) PCT Filed: Nov. 8, 2011
(86) PCT No.: PCT/JP2011/076132

§ 371 Date: May 8, 2013

(30) Foreign Application Priority Data
Nov. 8, 2010 (JP) 2010-249876
Jul. 12, 2011 (JP) 2011-153862

(51) Int. Cl. A61K 47/48; G01N 33/82; C07K 16/28

(52) U.S. Cl. CPC A61K 47/48561; C07K 16/28; G01N 33/82
USPC 435/188; 530/389.6; 530/391.7; 530/388.22; 530/387.3

(57) ABSTRACT

It is intended to ameliorate arteriosclerosis or arteriosclerotic disease through a pharmacological mechanism that reduces the size of the arteriosclerotic lesion. The agent of the present invention comprises a complex comprising an antibody binding to folate receptor β (FRβ) and a cytotoxin or a cytotoxic agent conjugated with the antibody, or the antibody as an active ingredient.

### TECHNICAL FIELD

The present invention relates to a therapeutic agent for arteriosclerosis that ameliorates symptoms of arteriosclerosis, particularly, associated with an atheromatous plaque, and a therapeutic agent for arteriosclerotic disease that ameliorates arteriosclerotic disease, i.e., various diseases or symptoms associated with arteriosclerosis.

### BACKGROUND ART

Arteriosclerosis refers to the thickening and stiffening of the artery and is classified into atherosclerosis, arteriolosclerosis, and medial calcific sclerosis, etc. Particularly, atherosclerosis refers to the thickening and stiffening of the artery resulting from an atheromatous plaque on the inner wall of the artery. Also, arteriosclerotic disease encompasses various diseases associated with arteriosclerosis. Examples of the arteriosclerotic disease can include: cerebral infarction and cerebral hemorrhage associated with arteriosclerosis in the cerebral artery; ischemic heart disease, such as myocardial infarction and angina pectoris, associated with arteriosclerosis in the coronary artery; aortic aneurysm and aortic dissection associated with arteriosclerosis in the aorta; nephrosclerosis and eventual renal failure associated with arteriosclerosis in the renal artery; and arteriosclerosis obliterans associated with arteriosclerosis in the peripheral artery.

The current treatment of arteriosclerosis or arteriosclerotic disease lacks a therapeutic agent having the drug effect of reducing the size of the atheromatous plaque or stabilizing the atheromatous plaque, and places a high priority on the amelioration of risk factors (hyperlipidemia, hypertension, obesity, and diabetes mellitus). Also, the treatment of arteriosclerosis adopts surgical procedures, for example, “catheterization surgery” which is performed by inserting a thin tubular tool called catheter from a blood vessel, and “bypass surgery” which creates a blood vessel to bypass a blood vessel with arteriosclerosis.

Arteriosclerosis is considered to be formed by a process in which: LDL that has invaded the intima of a blood vessel undergoes oxidative modification; the thus-oxidized LDL is taken up by macrophages via scavenger receptors to form foamy cells; and the resulting foamy macrophages accumulate, thereby forming an atheromatous plaque.

Patent Literature 1 discloses a therapeutic agent for solid cancer targeting “cancer-related macrophages localized in cancer tissues”, the macrophages playing a central role in inflammatory reaction involved in cancer growth or metastasis. Patent Literature 1 also discloses, as one example of the therapeutic agent for solid cancer, use of a complex comprising an antibody binding to folate receptor β (FRβ) and a cytotoxin or a cytotoxic agent conjugated with the antibody. This is based on the finding that FRβ is expressed in the cancer-related macrophages localized in cancer tissues, but is hardly expressed in normal sites.

Alternatively, Patent Literature 2 and Non Patent Literatures 1, 2, and 3 have reported that: a gene encoding the antigen recognition site of an anti-human FRβ mouse monoclonal antibody is fused with a genetically modified Pseudomonas aeruginosa exotoxin gene to prepare a recombinant-type immunotoxin; and the selective removal of FRβ-expressing macrophages using the immunotoxin in a rheumatoid arthritis synovium SCID mouse model is effective for the treatment of rheumatoid arthritis or suppresses the differentiation of macrophages into osteoclasts or angiogenesis found in rheumatoid arthritis.

### CITATION LIST

Patent Literature 1: JP Patent Publication (Kokai) No. 2010-077026 A (2010)
Patent Literature 2: International Publication No. WO 2005/103250
Non Patent Literature 1: Nakashima-Matsushita N, Homma T, Yu S, Matsuda T, Sunahara N, Nakamura T, Tsukano M, Ratnam M, Matsuyama T. Selective expression of folate receptor beta and its possible role in methotrexate transport in synovial macrophages from patients with rheumatoid arthritis. Arthritis Rheum. 1999 August; 42 (8): 1609-16.
Non Patent Literature 2: Nagayoshi R, Nagai T, Matsushita K, Sato K, Sunahara N, Matsuda T, Nakamura T, Komiya S, Onda M, Matsuyama T. Effectiveness of anti-folate receptor beta antibody conjugated with truncated Pseudomonas exotoxin in the targeting of rheumatoid arthritis synovial macrophages. Arthritis Rheum. 2005 September; 52 (9): 2666-75.
Non Patent Literature 3: Nagai T, Tanaka M, Tsuneyoshi Y, Matsushita K, Sunahara N, Matsuda T, Yoshida H, Komiya S, Onda M, Matsuyama T. In vitro and in vivo efficacy of a recombinant immunotoxin against folate receptor beta on the activation and proliferation of rheumatoid arthritis synovial cells. Arthritis Rheum. 2006 October; 54 (10): 3126-34.

### SUMMARY OF INVENTION

Thus, in light of the present circumstances as described above, an object of the present invention is to provide a therapeutic agent for arteriosclerosis and a therapeutic agent for arteriosclerotic disease, which can ameliorate arteriosclerosis or arteriosclerotic disease through a pharmacological mechanism that reduces the size of the arteriosclerotic lesion. Another object of the present invention is to provide a diagnostic agent for arteriosclerosis or arteriosclerotic disease that detects the arteriosclerotic lesion with folate receptor β (FRβ) as an index.

The present inventors have completed the present invention by finding that: FRβ is expressed in an activated macrophage present in an arteriosclerotic lesion; and the size of the arteriosclerotic lesion can be reduced or the arteriosclerotic lesion can be stabilized by targeting the activated macrophage.

The present invention encompasses the followings:

(1) A therapeutic agent for arteriosclerosis or arteriosclerotic disease, comprising a complex comprising an antibody binding to folate receptor β (FRβ) and a cytotoxin or a cytotoxic agent conjugated with the antibody, or the antibody as an active ingredient.
(2) A diagnostic agent for arteriosclerosis or arteriosclerotic disease, comprising an antibody binding to folate receptor β (FRβ) as an active ingredient.

In the present invention, examples of the arteriosclerosis can include atherosclerosis. In the present invention, examples of the arteriosclerotic disease can include one selected from the group consisting of cerebral infarction, cerebral hemorrhage, ischemic heart disease, aortic aneurysm, aortic dissection, nephrosclerosis, renal failure, and arteriosclerosis obliterans.

In the therapeutic agent according to the present invention, the complex can be a recombinant immunotoxin. In the therapeutic agent according to the present invention, the cytotoxin can be selected from the group consisting of Pseudomonas aeruginosa exotoxin, ricin A chain, deglycosylated ricin A chain, ribosome inactivating protein, alpha-sarcin, gelonin, aspergillin, restrictocin, ribonuclease, epipodophyllotoxin, and diphtheria toxin.

In the present invention, the antibody can be a chimeric antibody, a humanized antibody, or a human antibody. In the present invention, preferably, the antibody does not bind to folate receptor a. More specifically, the antibody is preferably an antibody directed against a polypeptide consisting of the amino acid sequence represented by SEQ ID NO: 1 or 2 or a partial peptide thereof consisting of 7 or more consecutive amino acids in the amino acid sequence represented by SEQ ID NO: 1 or 2. In this context, the antibody may be any of a monoclonal antibody, a polyclonal antibody, their antibody fragments, and a recombinant antibody. More specifically, the antibody preferably comprises an amino acid sequence comprising at least one complementarity determining region (CDR) in the respective amino acid sequences of any heavy chain (H chain) variable region and/or any light chain (L chain) variable region of an anti-human folate receptor β mouse monoclonal antibody or an anti-mouse folate receptor β rat monoclonal antibody.

Examples of the antibody can include an antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 3;
(b) a polynucleotide that comprises the deletion, substitution, addition, or insertion of one to several bases in the nucleotide sequence represented by SEQ ID NO: 3 and encodes a protein having the biological activity of binding to FRβ;
(c) a polynucleotide that has at least 90% sequence identity to the nucleotide sequence represented by SEQ ID NO: 3 and encodes a protein having the biological activity of binding to FRβ; and
(d) a polynucleotide that hybridizes under stringent conditions to a sequence complementary to the nucleotide sequence represented by SEQ ID NO: 3 and encodes a protein having the biological activity of binding to FRβ.

Examples of the antibody can include an antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 4;
(b) a polynucleotide that comprises the deletion, substitution, addition, or insertion of one to several bases in the nucleotide sequence represented by SEQ ID NO: 4 and encodes a protein having the biological activity of binding to FRp;
(c) a polynucleotide that has at least 90% sequence identity to the nucleotide sequence represented by SEQ ID NO: 4 and encodes a protein having the biological activity of binding to FRβ; and
(d) a polynucleotide that hybridizes under stringent conditions to a sequence complementary to the nucleotide sequence represented by SEQ ID NO: 4 and encodes a protein having the biological activity of binding to FRβ.

Examples of the antibody can include an antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 5;
(b) a polynucleotide that comprises the deletion, substitution, addition, or insertion of one to several bases in the nucleotide sequence represented by SEQ ID NO: 5 and encodes a protein having the biological activity of binding to FRβ;
(c) a polynucleotide that has at least 90% sequence identity to the nucleotide sequence represented by SEQ ID NO: 5 and encodes a protein having the biological activity of binding to FRβ; and
(d) a polynucleotide that hybridizes under stringent conditions to a sequence complementary to the nucleotide sequence represented by SEQ ID NO: 5 and encodes a protein having the biological activity of binding to FRβ.

Examples of the antibody can include an antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 6;
(b) a polynucleotide that comprises the deletion, substitution, addition, or insertion of one to several bases in the nucleotide sequence represented by SEQ ID NO: 6 and encodes a protein having the biological activity of binding to FRβ;
(c) a polynucleotide that has at least 90% sequence identity to the nucleotide sequence represented by SEQ ID NO: 6 and encodes a protein binding to FRβ; and
(d) a polynucleotide that hybridizes under stringent conditions to a sequence complementary to the nucleotide sequence represented by SEQ ID NO: 6 and encodes a protein having the biological activity of binding to FRβ.

Examples of the antibody can include an antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 7;
(b) a polynucleotide that comprises the deletion, substitution, addition, or insertion of one to several bases in the nucleotide sequence represented by SEQ ID NO: 7 and encodes a protein having the biological activity of binding to FRβ;
(c) a polynucleotide that has at least 90% sequence identity to the nucleotide sequence represented by SEQ ID NO: 7 and encodes a protein having the biological activity of binding to FRβ; and
(d) a polynucleotide that hybridizes under stringent conditions to a sequence complementary to the nucleotide sequence represented by SEQ ID NO: 7 and encodes a protein having the biological activity of binding to FRβ.

Examples of the antibody can include an antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 8;
(b) a polynucleotide that comprises the deletion, substitution, addition, or insertion of one to several bases in the nucleotide sequence represented by SEQ ID NO: 8 and encodes a protein having the biological activity of binding to FRβ;
(c) a polynucleotide that has at least 90% sequence identity to the nucleotide sequence represented by SEQ ID NO: 8 and encodes a protein having the biological activity of binding to FRβ; and
(d) a polynucleotide that hybridizes under stringent conditions to a sequence complementary to the nucleotide sequence represented by SEQ ID NO: 8 and encodes a protein having the biological activity of binding to FRβ.

Examples of the antibody can include an antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 9;
(b) a polynucleotide that comprises the deletion, substitution, addition, or insertion of one to several bases in the nucleotide sequence represented by SEQ ID NO: 9 and encodes a protein having the biological activity of binding to FRβ;
(c) a polynucleotide that has at least 90% sequence identity to the nucleotide sequence represented by SEQ ID NO: 9 and encodes a protein having the biological activity of binding to FRβ; and
(d) a polynucleotide that hybridizes under stringent conditions to a sequence complementary to the nucleotide sequence represented by SEQ ID NO: 9 and encodes a protein having the biological activity of binding to FRβ.

Examples of the antibody can include an antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 10;
(b) a polynucleotide that comprises the deletion, substitution, addition, or insertion of one to several bases in the nucleotide sequence represented by SEQ ID NO: 10 and encodes a protein having the biological activity of binding to FRβ;
(c) a polynucleotide that has at least 90% sequence identity to the nucleotide sequence represented by SEQ ID NO: 10 and encodes a protein binding to FRβ; and
(d) a polynucleotide that hybridizes under stringent conditions to a sequence complementary to the nucleotide sequence represented by SEQ ID NO: 10 and encodes a protein having the biological activity of binding to FRβ.

The antibody can be, for example, a single-chain or double-chain antibody comprising an H chain consisting of the polypeptide represented by SEQ ID NO: 11 and an L chain consisting of the polypeptide represented by SEQ ID NO: 12.

The antibody can be, for example, a single-chain or double-chain antibody comprising an H chain consisting of the polypeptide represented by SEQ ID NO: 13 and an L chain consisting of the polypeptide represented by SEQ ID NO: 14.

The present specification encompasses the contents described in the specifications and/or drawings of Japanese Patent Application Nos. 2010-249876 and 2011-153862 on which the priority of the present application is based.

### BRIEF DESCRIPTION OF DRAWINGS

FIG. 1 is a photograph showing results of immunochemical staining using ApoE-knockout mice. FIG. 1(a) shows the results of staining with an anti-mouse FRβ mouse monoclonal antibody. FIG. 1(b) shows the results of staining with an anti-mouse macrophage marker monoclonal antibody.

FIG. 2 is a photograph showing results of immunochemical staining. FIG. 2(a) shows the results of Oil Red O staining of an arteriosclerotic lesion in an immunotoxin-administered group. FIG. 2(b) shows the results of Oil Red O staining of an arteriosclerotic lesion in a control group. FIG. 2(c) shows the results of Oil Red O staining of an arteriosclerotic lesion in a placebo-administered group. FIG. 2(d) shows the results of Oil Red O staining of an arteriosclerotic lesion in an antibody-administered group.

FIG. 3 is a characteristic diagram showing results of quantifying arteriosclerotic lesions in an immunotoxin-administered group, a control group, a placebo-administered group, and an antibody-administered group.

FIG. 4 is a characteristic diagram showing results of measuring the number of peripheral blood monocytes in an immunotoxin-administered group, a control group, a placebo-administered group, and an antibody-administered group.

FIG. 5 is a characteristic diagram showing results of measuring total cholesterol levels in blood in an immunotoxin-administered group, a control group, a placebo-administered group, and an antibody-administered group.

FIG. 6-1 is a photograph showing results of immunostaining of an arteriosclerotic lesion in a mouse of an immunotoxin-administered group, a control group, a placebo-administered group, or an antibody-administered group obtained in 28th day after the completion of administration. FIG. 6-1(a) shows the results of immunostaining in the control group. FIG. 6-1(b) shows the results of immunostaining in the immunotoxin-administered group.

FIG. 6-2 is a photograph showing results of immunostaining of an arteriosclerotic lesion in a mouse of an immunotoxin-administered group, a control group, a placebo-administered group, or an antibody-administered group obtained in 28th day after the completion of administration. FIG. 6-2(c) shows the results of immunostaining in the placebo-administered group. FIG. 6-2(d) shows the results of immunostaining in the antibody-administered group.

FIG. 7 is a characteristic diagram showing results of counting the number of FRβ-expressing cells in an arteriosclerotic lesion in a mouse aortic root of an immunotoxin-administered group, a control group, a placebo-administered group, or an antibody-administered group obtained in 28th day after the completion of administration.

FIG. 8 is a photograph showing results of immunochemical staining of a human carotid artery tissue.

FIG. 9 is a photograph showing results of detecting an arteriosclerotic lesion (arteriosclerotic lesion site) by molecular imaging using a fluorescent label.

### DESCRIPTION OF EMBODIMENTS

Hereinafter, the therapeutic agent and the diagnostic agent for arteriosclerosis or arteriosclerotic disease according to the present invention will be described in detail.

The therapeutic agent of the present invention comprises a complex comprising an antibody binding to folate receptor β (FRβ) and a cytotoxin or a cytotoxic agent conjugated with the antibody, or the antibody as an active ingredient. Specifically, the therapeutic agent of the present invention comprises any one or both of the complex and the antibody as an active ingredient. Also, the diagnostic agent of the present invention comprises an antibody binding to folate receptor β (FRβ) as an active ingredient. The “folate receptor β” or “FRβ” used in the present specification means a receptor βprotein that is expressed on the cell surface of an activated macrophage present in the arteriosclerotic lesion of a mammal. The mammal includes primates including humans, livestock animals such as cattle, pigs, horses, goats, and sheep, and pet animals such as dogs and cats. The mammal is preferably a human.

The “antibody binding to folate receptor β (FRβ)” used in the present specification refers to an antibody capable of recognizing the FRβ protein and binding to the protein. As described below, the antibody may be an intact antibody or may be an antibody fragment or a synthetic antibody (e.g. a recombinant antibody, a bispecific antibody, a chimeric antibody, or a humanized antibody) as long as the antibody has binding affinity for the activated macrophage. When FRβ is expressed on the surface of a cell other than the activated macrophage present in an arteriosclerotic lesion, these antibodies are also capable of binding to both the activated macrophage and the cell. In the case of using the antibody for a human, the antibody is preferably a human antibody or a humanized antibody.

The “cytotoxin” or the “cytotoxic agent” used in the present specification refers to any substance having the ability to kill or damage the activated macrophage.

### 1. Antibody

In the present invention, the antibody specifically binds to FRβ on an activated macrophage present in an arteriosclerotic lesion. In this context, the term “specifically” means that the antibody binds to FRβ on the macrophage through immunological reaction, but does not substantially bind to a protein other than FRβ or a protein having 80% or more sequence identity thereto. In this respect, desirably, the antibody does not bind to FRα (e.g., human FRα has approximately 70% amino acid sequence identity to human FRβ (JP Patent Publication (Kohyo) No. 2008-500025 A (2008))), which is an isoform of FR.

The antibody that can be used in the present invention is the whole antibody molecule capable of binding to the antigen FRβ protein or a partial peptide thereof, or a fragment of the antibody. The partial peptide has 5 or more, preferably 7 or more, more preferably 8 or more consecutive amino acids. In general, an antigenic epitope or an antigen determinant consists of approximately 5 to approximately 10 amino acids and has a consecutive or non-consecutive amino acid sequence.

The antibody of the present invention may be any of a monoclonal antibody, a polyclonal antibody, a human antibody, and their antibody fragments as long as the antibody binds, preferably specifically, to FRβ.

Also, the antibody of the present invention may belong to any immunoglobulin (Ig) class (IgA, IgG, IgE, IgD, IgM, etc.) and subclass (IgG1, IgG2, IgG3, IgG4, IgA1, IgA2, etc.). The immunoglobulin may have κ or λ light chains.

The antibody fragment of the present invention includes, for example, Fab, Fab′, F(ab′)₂, Fv, a heavy chain monomer or dimer, a light chain monomer or dimer, and a dimer consisting of one heavy chain and one light chain. A method for preparing such a fragment is known in the art. For example, the antibody fragment of the present invention can be obtained by the digestion of the antibody molecule with protease such as papain or pepsin or by a genetic engineering approach known in the art.

The antibody of the present invention may also be a recombinant antibody, a chimeric antibody, a humanized antibody, or the like. The recombinant antibody includes, for example, a single-chain antibody (scFv) and a bispecific antibody. The bispecific antibody refers to an antibody having two different binding specificities and includes, for example, diabody, single chain diabody (ScDb), and dsFv-dsFv (see Ryutaro Asano, The Journal of Biochemistry 77 (12), 1497-1500, 2005).

Hereinafter, a method for preparing the antibody for use in the present invention will be described in detail.

In order to prepare the antibody that can be used in the present invention, first, a protein to be used as an immunogen (antigen), i.e., the FRβ protein or a partial peptide thereof, is prepared. In this context, the partial peptide has a sequence of 5 or more, preferably 7 or more consecutive amino acids. The origin of the FRβ protein that can be used as an immunogen is not particularly limited as long as the protein can direct the antibody capable of specifically binding to the targeted FRβ. For example, FRβ protein or a partial peptide thereof derived from a mammal such as a human or a mouse is used as an immunogen. In the present invention, human FRβ protein consisting of the amino acid sequence represented by SEQ ID NO: 1 or a partial peptide thereof, or mouse FRβ protein consisting of the amino acid sequence represented by SEQ ID NO: 2 or a partial peptide thereof can be used as an immunogen. The human FRβ protein consisting of the amino acid sequence represented by SEQ ID NO: 1 or the partial peptide thereof is particularly preferably used as an immunogen.

This FRβ protein or partial peptide thereof can be prepared by an approach known in the art, for example, a solid-phase peptide synthesis method, based on FRβ amino acid sequence information (e.g., SEQ ID NO: 1). Sequence information about FRβ derived from other mammals including humans is available from, for example, GenBank (NCBI, USA) and EMBL (EBI, Europe).

Alternatively, the FRβ protein or the partial peptide thereof may be produced using a gene recombination approach. In brief, a DNA sequence encoding the FRβ protein is ligated with an appropriate vector for protein production, which is then introduced into a host so that the target FRβ protein or the partial peptide thereof can be expressed. The resulting host can produce the FRβ protein or the partial peptide thereof. This approach is well known by those skilled in the art. Those skilled in the art can appropriately select the vector, the host cells, a transformation method, a culture method, and a target protein purification method adopted in this case. For the gene recombination approach, see, for example, Sambrook et al., Molecular Cloning: A Laboratory Manual, 2nd ed., Cold Spring Harbor Laboratory Press (1989), and Ausubel et al., Current Protocols in Molecular Biology, John Wiley & Sons (1998).

The FRβ protein or the partial peptide thereof thus prepared can be used as an immunogen to produce the antibody of the present invention. Alternatively, an expression vector having a DNA insert encoding the target FRβ protein or the partial peptide thereof, or a mammalian cell expressing the protein or the partial peptide thereof may be used as an immunogen to produce the antibody of the present invention.

The polyclonal antibody of the present invention can be produced from the antiserum of a mammal, for example, a rabbit, a rat, or a mouse, immunized with the immunogen thus prepared. Specifically, the immunogen, if necessary together with an adjuvant for enhancing immunogenicity, is administered intravenously, subcutaneously, or intraperitoneally to the mammal. For example, a commercially available complete or incomplete Freund's adjuvant, aluminum hydroxide, alum, or muramyl peptide (one type of bacterial cell wall-related peptide) can be used as the adjuvant. Then, the mammal is immunized once to seven times at intervals of a few days to a few weeks. One to seven days after the final immunization day, antibody titers are measured by, for example, enzyme immunoassay such as ELISA. At the day when the animal exhibits the largest antibody titer, blood is collected therefrom to obtain antiserum. The antiserum thus obtained may be used directly or may be used after being purified once or several times using a column with the FRβ protein or the partial peptide thereof immobilized thereon.

The monoclonal antibody that can be used in the present invention can be prepared as follows: hybridomas are prepared from antibody-producing cells (e.g., spleen-derived lymphocytes or lymphoid cells) obtained from the mammal thus sensitized by immunization and myeloma cells having no ability to produce autoantibodies, and the hybridomas are cloned. A clone producing a monoclonal antibody that exhibits specific affinity for the antigen used in the immunization can be selected to produce the monoclonal antibody of the present invention. The method for producing such hybridomas is well known in the art and can be performed according to, for example, the method of Kohler and Milstein et al. (Nature (1975) 256: 495-96).

Examples of the monoclonal antibody of the present invention can include monoclonal antibodies produced by a mouse-mouse hybridoma clone 36b or clone 94b obtained by the fusion between the splenic cell of a mouse immunized with human FRβ-expressing cells and a mouse myeloma cell.

Other examples of the monoclonal antibody of the present invention can include monoclonal antibodies produced by a rat-rat hybridoma clone CL5 or clone CL10 obtained by the fusion between the splenic cell of a rat immunized with mouse FRβ-expressing cells and a rat myeloma cell.

The present invention encompasses a gene that comprises a nucleic acid derived from any of the hybridomas thus prepared and encodes an antibody comprising an H chain or an L chain of the monoclonal antibody produced by any of the hybridomas. This nucleic acid can be obtained by a usual genetic engineering approach from the hybridoma. Also, its nucleotide sequence can be determined by a sequencing method known in the art. As an example, the nucleotide sequences of the H chain and L chain variable region genes of the monoclonal antibody produced by the mouse-mouse hybridoma clone 36b cell are shown in SEQ ID NOs: 3 and 4, respectively. Also, the nucleotide sequences of the H chain and L chain variable region genes of the monoclonal antibody produced by the mouse-mouse hybridoma clone 94b cell are shown in SEQ ID NOs: 5 and 6, respectively. As another example, the nucleotide sequences of the H chain and L chain variable region genes of the monoclonal antibody produced by the rat-rat hybridoma clone CL5 are shown in SEQ ID NOs: 7 and 8, respectively. Also, the nucleotide sequences of the H chain and L chain variable region genes of the monoclonal antibody produced by the rat-rat hybridoma clone CL10 are shown in SEQ ID NOs: 9 and 10, respectively.

The gene of the present invention is not limited to these H chain variable region genes (e.g., SEQ ID NOs: 3, 5, 7, and 9) and L chain variable region genes (e.g., SEQ ID NOs: 4, 6, 8, and 10) of the monoclonal antibodies produced by the hybridomas prepared as described above, and encompasses variants of these genes. Examples of such variants can include the followings:

(i) a variant that has the deletion, substitution, addition, or insertion of one to several bases in any of the H chain or L chain variable region genes and encodes a protein having the biological activity of binding to FRβ with grade or quality comparable to that of the H chain or L chain variable region; (ii) a variant that has a nucleotide sequence substantially identical to any of the H chain variable region-encoding nucleic acids or the L chain variable region-encoding nucleic acids and encodes a protein having the biological activity of binding to FRβ with grade or quality comparable to that of the H chain or L chain variable region; and (iii) a variant that hybridizes under stringent conditions to a sequence complementary to any of the H chain variable region-encoding nucleic acids or the L chain variable region-encoding nucleic acids and encodes a protein having the biological activity of binding to FRGS with grade or quality comparable to that of the H chain or L chain variable region.

The term “several” used in relation to the H chain and L chain variable region genes of the present invention refers to 1 to 20, preferably 1 to 15, more preferably 1 to 10.

The term “substantially identical” used in relation to the H chain and L chain variable region genes of the present invention means having at least 80%, preferably at least 85%, more preferably at least 90%, further preferably at least 95%, 96%, 97%, 98%, or 99% identity to any of the H chain variable region genes (e.g., SEQ ID NOs: 3, 5, 7, and 9) or the L chain variable region genes (e.g., SEQ ID NOs: 4, 6, 8, and 10) of the monoclonal antibodies produced by the hybridomas prepared as described above. In this context, % identity between two sequences is a function of the number of identical positions shared by the sequences (i.e., % identity=the number of identical positions/the total number of positions (e.g., partially overlapping positions)×100).

The % identity between two sequences can be determined using, for example, the algorithm of Karlin and Altschul (1990) (Proc. Natl. Acad. Sci. USA 87: 2264) or its modification (1993) (Karlin and Altschul, Proc. Natl. Acad. Sci. USA 90: 5873-5877). This type of algorithm is incorporated in the NBLAST and XBLAST program of Altschul et al. (1990) J. Mol. Biol. 215: 403. Also, Gapped BLAST described in Altschul et al. (1997) Nucleic Acids Res. 25: 3389 can be used in order to obtain gap-containing alignment for comparison. Alternatively, PSI-BLAST may be used in order to perform iterative search that detects the distant relationship between molecules. In the case of using the BLAST, Gapped BLAST, and PSI-BLAST programs, the default parameters of their respective programs (e.g., XBLAST and NBLAST) can be used (see http://www.ncbi.nlm.nih.gov.). An alternative preferable example of the algorithm that can be used for sequence comparison is the algorithm of Myers and Miller (1988), CABIOS 4: 11-17. This type of algorithm is incorporated in the ALIGN program (version 2.0) included in the GCC sequence aligning software package.

The “stringent conditions” used in the present specification involve, but not limited to, for example, hybridization at 30° C. to 50° C. for 1 to 24 hours in 3 to 4×SSC (150 mM sodium chloride and 15 mM sodium citrate, pH 7.2) and 0.1 to 0.5% SDS, more preferably hybridization at 40° C. to 45° C. for 1 to 24 hours in 3.4×SSC and 0.3% SDS, followed by washing Examples of the washing conditions can include conditions involving continuous washing at room temperature using a solution containing 2×SSC and 0.1% SDS, a 1×SSC solution, and a 0.2×SSC solution. However, these combinations for the conditions are provided for illustrative purposes, and those skilled in the art can achieve stringency similar to above by appropriately combining the above-described and/or other factors (e.g., the concentration, length, and GC content of a hybridization probe, and hybridization reaction time) that determine hybridization stringency.

The “comparable grade” used in the present specification means that, for example, biological activity such as binding specificity or binding affinity for the FRβ antigen is substantially identical to that of the H chain or L chain variable region. This term may include activity with quality substantially comparable to that of the H chain or L chain variable region. In this context, the activity with “comparable quality” means that the nature of activity such as specific binding activity against the FRβ antigen is identical to that of the H chain or L chain variable region or physiological, pharmacological, or biological properties are identical to those of the H chain or L chain variable region.

Other examples of these “stringent conditions” for hybridization are described in, for example, Sambrook et al. (supra) and Ausubel et al. (supra). The conditions described in these literatures may be used in the present invention.

The variant may be a naturally occurring variant or may result from artificial mutagenesis. The artificial mutagenesis can be performed by a routine method using, for example, site-directed mutagenesis (Proc Natl Acad Sci USA., 1984, 81: 5662; and Sambrook et al. (supra)).

In the present invention, a recombinant antibody may be prepared using a gene recombination technique based on the hybridomas prepared as described above. Specifically, a gene encoding the monoclonal antibody is cloned from any of the prepared hybridomas and inserted into an appropriate vector, which is then introduced into a host, for example, a mammalian cell line such as Chinese hamster ovary (CHO) cells, E. coli, yeast cells, insect cells, or plant cells. The resulting host can produce the recombinant antibody (P. J. Delves., ANTIBODY PRODUCTION ESSENTIAL TECHNIQUES., 1997 John Wiley & Sons; P. Shepherd and C. Dean., Monoclonal Antibodies., 2000 OXFORD UNIVERSITY PRESS; and J. W. Goding., Monoclonal Antibodies: principles and practice., 1993 ACADEMIC PRESS).

Alternatively, a transgenic mouse, cattle, goat, sheep, or pig having the gene of the antibody of interest incorporated in endogenous genes is prepared using a transgenic animal preparation technique and immunized with the FRβ protein or the partial peptide thereof as an antigen. Then, an antibody derived from the antibody gene can also be obtained in large amounts from the blood, milk, or the like of the transgenic animal. Among these transgenic animals, a human antibody-producing animal (e.g., mouse or cattle) that has no endogenous antibody gene and retains a human antibody gene is also known. Use of such an animal can yield a complete human antibody binding to human FRβ (e.g., International Publication Nos. WO 96/9634096, WO 96/33735, and WO 98/24893). In addition, hybridomas prepared from the antibody-producing cells (e.g., B cells) of the animal and myeloma cells can also be cultured in vitro and allowed to produce the monoclonal antibody by the approach as described above. In this case, the hybridomas are grown, maintained, and stored according to various conditions such as the properties of the cell type to be cultured, the purpose of tests or research, and a culture method, and the hybridoma culture can be carried out using a known nutrient medium as used for producing monoclonal antibodies into a culture supernatant, or using every nutrient medium prepared by induction from a known basal medium.

The produced monoclonal antibody can be purified by appropriately combining methods well known in the art, for example, chromatography using protein A or protein G columns, ion-exchange chromatography, hydrophobic chromatography, ammonium sulfate precipitation, gel filtration, and affinity chromatography.

The antibody that can be used in the present invention may be a chimeric antibody. The chimeric antibody of the present invention can be produced using a technique described in, for example, Morrison et al., 1984, Proc. Natl. Acad. Sci., 81: 6851-6855; Neuberger et al., 1984, Nature, 312: 604-608; and Takeda et al., 1985, Nature, 314: 452-454. In these techniques, a gene from a mouse antibody molecule having appropriate antigen specificity is spliced with a gene from a human antibody molecule having appropriate biological activity. The chimeric antibody refers to a molecule containing different portions derived from different animal species. Examples of the chimeric antibody can include an antibody having the H chain and/or L chain variable regions of an anti-FRβ mouse or rat monoclonal antibody and immunoglobulin constant regions derived from a mammal different therefrom.

Alternative examples of the antibody according to the present invention can include a “humanized antibody”, which is an antibody having a portion of mouse or rat mAb-derived variable regions or hypervariable region-containing variable regions and human immunoglobulin constant regions or a portion of human immunoglobulin variable regions and human immunoglobulin constant regions. Desirably, the humanized antibody has less than approximately 10% mouse-derived antibody domains.

The humanized antibody can comprise, for example, an amino acid sequence comprising at least one complementarity determining region (CDR1, CDR2, or CDR3) in the respective amino acid sequences of H chain and/or L chain variable regions of an anti-human FRβ mouse monoclonal antibody (or an anti-mouse FRβ rat monoclonal antibody). More specific examples thereof can include the following antibodies:

(1) An antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 3;
(b) a polynucleotide that comprises the deletion, substitution, addition, or insertion of one to several bases in the nucleotide sequence represented by SEQ ID NO: 3 and encodes a protein having the biological activity of binding to FRp;
(c) a polynucleotide that has at least 90% sequence identity to the nucleotide sequence represented by SEQ ID NO: 3 and encodes a protein having the biological activity of binding to FRβ; and
(d) a polynucleotide that hybridizes under stringent conditions to a sequence complementary to the nucleotide sequence represented by SEQ ID NO: 3 and encodes a protein having the biological activity of binding to FRβ.

(2) An antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 4;
(b) a polynucleotide that comprises the deletion, substitution, addition, or insertion of one to several bases in the nucleotide sequence represented by SEQ ID NO: 4 and encodes a protein having the biological activity of binding to FRβ;
(c) a polynucleotide that has at least 90% sequence identity to the nucleotide sequence represented by SEQ ID NO: 4 and encodes a protein having the biological activity of binding to FRβ; and
(d) a polynucleotide that hybridizes under stringent conditions to a sequence complementary to the nucleotide sequence represented by SEQ ID NO: 4 and encodes a protein having the biological activity of binding to FRβ.

(3) An antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 5;
(b) a polynucleotide that comprises the deletion, substitution, addition, or insertion of one to several bases in the nucleotide sequence represented by SEQ ID NO: 5 and encodes a protein having the biological activity of binding to FRβ;
(c) a polynucleotide that has at least 90% sequence identity to the nucleotide sequence represented by SEQ ID NO: 5 and encodes a protein having the biological activity of binding to FRβ; and
(d) a polynucleotide that hybridizes under stringent conditions to a sequence complementary to the nucleotide sequence represented by SEQ ID NO: 5 and encodes a protein having the biological activity of binding to FRβ.

(4) An antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 6;
(b) a polynucleotide that comprises the deletion, substitution, addition, or insertion of one to several bases in the nucleotide sequence represented by SEQ ID NO: 6 and encodes a protein having the biological activity of binding to FRβ;
(c) a polynucleotide that has at least 90% sequence identity to the nucleotide sequence represented by SEQ ID NO: 6 and encodes a protein binding to FRβ; and
(d) a polynucleotide that hybridizes under stringent conditions to a sequence complementary to the nucleotide sequence represented by SEQ ID NO: 6 and encodes a protein having the biological activity of binding to FRβ.

(5) An antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 7;
(b) a polynucleotide that comprises the deletion, substitution, addition, or insertion of one to several bases in the nucleotide sequence represented by SEQ ID NO: 7 and encodes a protein having the biological activity of binding to FRβ;
(c) a polynucleotide that has at least 90% sequence identity to the nucleotide sequence represented by SEQ ID NO: 7 and encodes a protein having the biological activity of binding to FRβ; and
(d) a polynucleotide that hybridizes under stringent conditions to a sequence complementary to the nucleotide sequence represented by SEQ ID NO: 7 and encodes a protein having the biological activity of binding to FRβ.

(6) An antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 8;
(b) a polynucleotide that comprises the deletion, substitution, addition, or insertion of one to several bases in the nucleotide sequence represented by SEQ ID NO: 8 and encodes a protein having the biological activity of binding to FRβ;
(c) a polynucleotide that has at least 90% sequence identity to the nucleotide sequence represented by SEQ ID NO: 8 and encodes a protein having the biological activity of binding to FRβ; and
(d) a polynucleotide that hybridizes under stringent conditions to a sequence complementary to the nucleotide sequence represented by SEQ ID NO: 8 and encodes a protein having the biological activity of binding to FRβ.

(7) An antibody comprising an amino acid sequence comprising at least one complementarity determining region (CDR) in the amino acid sequence of an H chain variable region encoded by the following polynucleotide (a), (b), (c), or (d):

(a) a polynucleotide consisting of the nucleotide sequence represented by SEQ ID NO: 9;
(b) a polynucleotide that comprises the deletion, substitution, addition, or insertion of one to several bases in the nucleotide sequence represented by SEQ ID NO: 9 and encodes a protein having the biological activity of binding to FRβ;
(c) a polynucleotide that has at least 90% sequence identity to the nucleotide sequence represented by SEQ ID NO: 9 and encodes a protein having the biological activity of binding to FRβ; and
(d) a polynucleotide that hybridizes under stringent conditions to a sequence complementary to the nucleotide sequence represented by SEQ ID NO: 9 and encodes a protein having the biological activity of binding to FRβ.

(8) An antibody comprising an amino acid sequence comprising at least one complementarity determining region (CDR) in the amino acid sequence of an L chain variable region encoded by the following polynucleotide (a), (b), (c), or (d):

(a) a polynucleotide consisting of the nucleotide sequence represented by SEQ ID NO: 10;
(b) a polynucleotide that comprises the deletion, substitution, addition, or insertion of one to several bases in the nucleotide sequence represented by SEQ ID NO: 10 and encodes a protein having the biological activity of binding to FRβ;
(c) a polynucleotide that has at least 90% sequence identity to the nucleotide sequence represented by SEQ ID NO: 10 and encodes a protein binding to FRβ; and
(d) a polynucleotide that hybridizes under stringent conditions to a sequence complementary to the nucleotide sequence represented by SEQ ID NO: 10 and encodes a protein having the biological activity of binding to FRβ.

A human acceptor antibody sequence suitable for the mouse donor sequence may be identified by the computer comparison of the amino acid sequence of the mouse variable region with the H chain or L chain sequences of known human antibodies. Variable domains from a human antibody whose framework sequences have high sequence identity to the framework regions of the mouse L chain and H chain variable regions can be identified by referring to the Kabat database offered by NCBI BLAST (USA) using the mouse framework sequences. In this regard, an acceptor sequence sharing 80% or more, preferably 90% or more sequence identity to the mouse donor sequence can be selected. A human acceptor antibody sequence suitable for the rat donor sequence can also be identified in the same way as above.

On the basis of nucleotide sequences encoding the human acceptor antibody H chain and L chain sequences thus identified, gene recombination is performed so that a portion of the variable regions is replaced by the corresponding sites of the mouse antibody. The obtained DNAs encoding the human/mouse chimeric H chain and L chain are incorporated into expression vectors, with which appropriate host cells can then be transformed to clone and produce the humanized antibody.

The chimeric antibody or humanized antibody as described above can advantageously reduce antigenicity for its application to humans.

The antibody of the present invention may also be a single-chain antibody (scFv) against the FRβ protein or the partial peptide thereof prepared using a technique described in, for example, U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546. The single-chain antibody of the present invention can be formed, for example, by: preparing each of H chain and L chain fragments according to a routine method based on sequence information about the H chain variable regions (e.g., SEQ ID NOs: 3, 5, 7, and 9) and the L chain variable region genes (e.g., SEQ ID NOs: 4, 6, 8, and 10) of the monoclonal antibodies of the present invention; and linking the H chain and L chain fragments of Fv regions through amino acid bridge to obtain a single-chain polypeptide.

### 2. Complex

The complex serving as an active ingredient in the therapeutic agent of the present invention basically comprises: the antibody binding to FRβ as a molecular target expressed on the surface of an activated macrophage as described in the preceding paragraph 1; and a cytotoxin or a cytotoxic agent that causes the cell death of the macrophage (and, in some cases, other cells).

In this context, the cell death means the death, killing, or damage of the cell and is caused by the cytotoxin or the cytotoxic agent. The cytotoxin is a protein also named as so-called toxin, whereas the cytotoxic agent is a low-molecular-weight chemotherapeutic. The former includes toxins derived from microbes, particularly, bacteria. On the other hand, the latter includes alkylating agents, metabolic antagonists, antibiotics, molecular target drugs, vegetable alkaloids, hormone agents, and the like.

When the complex of the present invention comprises the antibody and the cytotoxin, these components can take the form of a fusion protein. In this case, the cytotoxin can bind to, preferably, the C terminus of the antibody protein, if necessary via a linker (e.g., peptide). On the other hand, when the complex of the present invention comprises the antibody and the cytotoxic agent, these components can be conjugated through a covalent or noncovalent bond via functional groups for the bond.

According to the present invention, the complex can recognize an activated macrophage present in an arteriosclerotic lesion and induce the cell death of the activated macrophage, resulting in the involution of the arteriosclerotic lesion.

### 2.1 Cytotoxin or Cytotoxic Agent

The cytotoxin that can be used in the present invention is any cytotoxin that can be used for the purpose of inducing the cell death of an activated macrophage present in an arteriosclerotic lesion. Examples thereof can include Pseudomonas aeruginosa exotoxin, ricin A chain, deglycosylated ricin A chain, ribosome inactivating protein, alpha-sarcin, gelonin, aspergillin, restrictocin, ribonuclease, epipodophyllotoxin, and diphtheria toxin. In the present invention, Pseudomonas aeruginosa exotoxin is particularly preferably used.

Alternatively, the cytotoxic agent that can be used in the present invention can be selected appropriately from alkylating agents, metabolic antagonists, antibiotics, molecular target drugs, vegetable alkaloids, hormone agents, and the like.

### 2.2 Complex Such as Recombinant Immunotoxin

In order to prepare a recombinant immunotoxin, which is a complex of the present invention, the antibody of the present invention or the partial peptide thereof prepared as described above is conjugated with a cytotoxin. Specifically, the recombinant immunotoxin according to the present invention is a chimeric molecule in which the antibody of the present invention binding to the target (i.e., FRβ protein) is conjugated with the cytotoxin or its subunit. In the present invention, a cytotoxin derived from a plant, a bacterium, or the like, a cytotoxin originating from a human, or a synthetic cytotoxin can be used.

The conjugation of the antibody of the present invention with the cytotoxin can be performed as follows: a reactive group (e.g., an amino, carboxyl, or hydroxy group) in the antibody molecule is used, and a cytotoxin having a functional group capable of reacting with the reactive group can be contacted with the antibody to obtain the recombinant immunotoxin of the present invention. Alternatively, a fusion protein of one of the H chain and L chain fragments with the cytotoxin may be prepared by a genetic engineering approach based on sequence information about the H chain variable region genes (SEQ ID NOs: 3, 5, 7, and 9) and the L chain variable region genes (SEQ ID NOs: 4, 6, 8, and 10) of the antibodies of the present invention. A single-chain antibody or a double-chain antibody can be formed from the fusion protein, together with the other fragment unfused with the cytotoxin, via an SH bond, to prepare the recombinant protein of the present invention. The linking between the H chain and L chain fragments via an SH bond can be achieved by the exposure of mercapto groups (—SH groups) using, for example, a reducing agent (e.g., (3-mercaptoethanol or dithiothreitol), followed by mixing of the fragments.

One example of the recombinant immunotoxin of the present invention includes a recombinant immunotoxin comprising the monoclonal antibody produced by the mouse-mouse hybridoma clone 36b or clone 94b cell and Pseudomonas aeruginosa exotoxin. The recombinant immunotoxin comprising the monoclonal antibody produced by the mouse-mouse hybridoma clone 36b and Pseudomonas aeruginosa exotoxin is, for example, a single-chain or double-chain antibody comprising an H chain consisting of the polypeptide represented by SEQ ID NO: 11 and an L chain consisting of the polypeptide represented by SEQ ID NO: 12. Also, the recombinant immunotoxin comprising the monoclonal antibody produced by the mouse-mouse hybridoma clone 94b and Pseudomonas aeruginosa exotoxin is, for example, a single-chain or double-chain antibody comprising an H chain consisting of the polypeptide represented by SEQ ID NO: 13 and an L chain consisting of the polypeptide represented by SEQ ID NO: 14.

Another example of the complex of the present invention is a conjugate of the antibody binding to FRβ and the cytotoxic agent. In general, the cytotoxic agent can bind to the constant region, preferably, the C terminus, of the antibody. The binding can be carried out by using the NH2 group, SH group, OH group, COOH group, or the like of the antibody protein and chemically binding the antibody with a cytotoxic agent having a functional group reactive with this group, if necessary via, for example, a hydrocarbon linker.

### 3. Therapeutic Agent

The antibody of the present invention or the complex of the present invention described above can target FRβ highly expressed specifically on an activated macrophage present in an arteriosclerotic lesion and induce the cell death of the macrophage. In this way, the antibody of the present invention or the complex (e.g., recombinant immunotoxin) of the present invention can significantly decrease the number of such activated macrophages present in an arteriosclerotic lesion, thereby causing the involution of the arteriosclerotic lesion. More specifically, the antibody of the present invention or the complex (e.g., recombinant immunotoxin) of the present invention can be allowed to act on the arteriosclerotic lesion to bring the thickening and stiffening of the artery back to a (nearly) normal state by the involution of the atheromatous plaque.

The antibody of the present invention or the complex of the present invention can be prepared into a therapeutic agent for arteriosclerosis or a therapeutic agent for arteriosclerotic disease comprising this antibody or complex as an active ingredient. Specifically, the therapeutic agent according to the present invention comprises a therapeutically effective amount of the complex or the antibody of the present invention. In this context, the “therapeutically effective amount” refers to an amount capable of conferring therapeutic effects as to the given symptom or usage and varies depending on various factors such as the sex, age, body weight, severity of disease of a subject with arteriosclerosis and/or arteriosclerotic disease to be treated, and an administration route. The therapeutically effective amount can include, for example, an amount of 30 μg or more, preferably 40 μg or more, per day in terms of the amount of the complex of the present invention to be administered to an adult of 60 kg according to the given usage.

The therapeutic agent according to the present invention may further comprise, in addition to the antibody of the present invention or the complex of the present invention, one or more of physiologically acceptable pharmaceutical additives, for example, a diluent, a preservative, a solubilizer, an emulsifier, an adjuvant, an antioxidant, a tonicity agent, an excipient, and a carrier. Alternatively, the therapeutic agent of the present invention may be a mixture with an additional drug known in the art to be effective for the treatment of arteriosclerosis or arteriosclerotic disease. Examples of such a drug can include, but not particularly limited to, blood cholesterol-lowering agents, for example: HMG-CoA reductase inhibitors such as pravastatin, simvastatin, fluvastatin, and atorvastatin; probucol preparations such as probucol; anion-exchange resin agents such as cholestyramine and colestimide; and fibrate preparations such as clinofibrate, bezafibrate, and fenofibrate. Examples of such drugs other than the blood cholesterol-lowering agents can also include antiplatelet drugs and drugs for hypertension, diabetes mellitus, or hyperuricemia.

The therapeutic agent according to the present invention can be formulated for oral administration or parenteral administration (i.e., intravenous or intramuscular administration) based on injection, for example, bolus injection or continuous injection. Also, the therapeutic agent according to the present invention may be formulated for arterial injection. The preparation for injection can be provided in a unit dosage form supplemented with a preservative in, for example, an ampule or a multi-dose container. Alternatively, the therapeutic agent of the present invention may be prepared as a freeze-dried powder for reconstitution before use with a suitable vehicle, for example, pyrogen-free sterilized water.

The possible administration route is, for example, an oral route and intravenous, intramuscular, intraarterial, subcutaneous, or intraperitoneal injection. Alternatively, the therapeutic agent of the present invention may be contacted directly with an arteriosclerotic lesion by administration to the arteriosclerotic lesion-containing artery of a patient though arterial injection.

Examples of targets to be treated using the therapeutic agent of the present invention can include arteriosclerosis and arteriosclerotic disease. In this context, the arteriosclerotic disease is not limited by any means as long as the arteriosclerotic disease is a disease or a symptom associated with arteriosclerosis. Examples of the arteriosclerotic disease can include any disease or symptom associated with arteriosclerosis and specifically include cerebral infarction, cerebral hemorrhage, ischemic heart disease, aortic aneurysm, aortic dissection, nephrosclerosis, renal failure, and arteriosclerosis obliterans.

A subject to which the therapeutic agent of the present invention is applied is not particularly limited and may be any of a healthy individual, a patient affected by arteriosclerosis, a patient affected by arteriosclerotic disease, a patient under treatment of arteriosclerosis and/or arteriosclerotic disease, a healthy individual considering the prevention of arteriosclerosis and/or arteriosclerotic disease, and the like. In addition, the subject is not limited to a human and may be a non-human mammal. Examples of the non-human mammal can include humans, mice, rats, monkeys, rabbits, dogs, and cats.

### 4. Diagnostic Agent

The diagnostic agent of the present invention comprises the antibody binding to FRβ as a molecular target expressed on the surface of an activated macrophage as described in the paragraph 1, as an active ingredient. This means that the antibody described in the paragraph 1 can be used in the diagnosis of arteriosclerosis and/or arteriosclerotic disease. Specifically, arteriosclerosis and/or arteriosclerotic disease can be diagnosed using the antibody described in the paragraph 1, i.e., the antibody specifically binding to FRβ on the activated macrophage. In this context, the diagnosis of arteriosclerosis and/or arteriosclerotic disease is more specifically meant to include the detection of an arteriosclerotic lesion (arteriosclerotic lesion site).

Further specifically, the arteriosclerotic lesion (arteriosclerotic lesion site) can be detected by the application of the antibody described in the paragraph 1 to molecular imaging involving visualizing the antibody on a scintigram by conjugation with a radioactive substance or visualizing the antibody by conjugation with a contrast medium for a magnetic resonance imaging apparatus (MRI) or a microbubble contrast medium for an ultrasonic diagnosis apparatus. Particularly, the activated macrophage expressing FRβ is present in an unstable and active arteriosclerotic lesion (also referred to as an unstable plaque) rich in lipid components. Thus, use of the antibody described in the paragraph 1 can detect this active arteriosclerotic lesion having the unstable plaque. In this context, the arteriosclerotic lesion is known to include two types of plaques: unstable and stable plaques (Libby P et al.: Nat Med 8; 1257-1262, 2002). Clinically, thrombus formation accompanying the rupture of a plaque responsible for cerebral infarction or myocardial infarction is considered to occur in the unstable plaque. Thus, if this unstable plaque can be differentiated from the stable plaque and detected, a site at a high risk of the thrombus formation accompanying the rupture of a plaque can be diagnosed.

The diagnostic agent according to the present invention can be formulated as a diagnostic agent comprising the antibody described in the paragraph 1 as an active ingredient. Specifically, the diagnostic agent according to the present invention comprises a diagnostically effective amount of the antibody. In this context, the “diagnostically effective amount” refers to an amount that permits diagnosis as to the given symptom or usage and varies depending on various factors such as the sex, age, body weight, severity of disease of a subject with arteriosclerosis and/or arteriosclerotic disease to be diagnosed, and an administration route. The diagnostically effective amount can include, for example, an amount of 30 μg or more, preferably 40 μg or more, per day in terms of the amount of the complex of the present invention to be administered to an adult of 60 kg according to the given usage.

The diagnostic agent according to the present invention may further comprise, in addition to the antibody described in the paragraph 1, one or more of physiologically acceptable pharmaceutical additives, for example, a diluent, a preservative, a solubilizer, an emulsifier, an adjuvant, an antioxidant, a tonicity agent, an excipient, and a carrier.

The diagnostic agent according to the present invention can be formulated for oral administration or parenteral administration (i.e., intravenous or intramuscular administration) based on injection, for example, bolus injection or continuous injection. Also, the diagnostic agent according to the present invention may be formulated for arterial injection. The preparation for injection can be provided in a unit dosage form supplemented with a preservative in, for example, an ampule or a multi-dose container. Alternatively, the diagnostic agent of the present invention may be prepared as a freeze-dried powder for reconstitution before use with a suitable vehicle, for example, pyrogen-free sterilized water.

The possible administration route is, for example, an oral route and intravenous, intramuscular, intraarterial, subcutaneous, or intraperitoneal injection. Alternatively, the diagnostic agent of the present invention may be contacted directly with an arteriosclerotic lesion by administration to the arteriosclerotic lesion-containing artery of a patient through arterial injection.

Examples of targets to be diagnosed using the diagnostic agent of the present invention can include arteriosclerosis and arteriosclerotic disease. In this context, the arteriosclerotic disease is not limited by any means as long as the arteriosclerotic disease is a disease or a symptom associated with arteriosclerosis. Examples of the arteriosclerotic disease can include any disease or symptom associated with arteriosclerosis and specifically include cerebral infarction, cerebral hemorrhage, ischemic heart disease, aortic aneurysm, aortic dissection, nephrosclerosis, renal failure, and arteriosclerosis obliterans.

A subject to which the diagnostic agent of the present invention is applied is not particularly limited and may be any of a healthy individual, a patient affected by arteriosclerosis, a patient affected by arteriosclerotic disease, a patient under treatment of arteriosclerosis and/or arteriosclerotic disease, a healthy individual considering the prevention of arteriosclerosis and/or arteriosclerotic disease, and the like. In addition, the subject is not limited to a human and may be a non-human mammal. Examples of the non-human mammal can include humans, mice, rats, monkeys, rabbits, dogs, and cats.

Hereinafter, the present invention will be described in more detail with reference to Examples. However, the technical scope of the present invention is not intended to be limited to them.

### Example 1

### Production of Anti-Human FRβ Mouse Monoclonal Antibody and Anti-Mouse FRβ Rat Monoclonal Antibody

### [Preparation of Cell Expressing Antigen FRβ]

Total RNA was extracted from the rheumatoid arthritis synovium or the Balb/c mouse liver using Trizol (Gibco BRL Life Technologies, Inc.) and a cDNA synthesis kit (Invitrogen Corp.) according to the instruction manuals included therein. Then, cDNA was synthesized therefrom using SuperScript plasmid System (Invitrogen Corp.) according to the instruction manual included therein. Next, 1 μl of the rheumatoid synovium or Balb/c mouse liver cDNA was each individually added to Bioneer PCR premix (Bioneer Inc.). A sense primer (human rheumatoid synovium: agaaagacatgggtctggaaatggatg (SEQ ID NO: 15); or mouse liver: tctagaaagacatggcctggaaacag (SEQ ID NO: 16)) and an antisense primer (human rheumatoid synovium: gactgaactcagccaaggagccagagtt (SEQ ID NO: 17); or mouse liver: cccaacatggatcaggaact (SEQ ID NO: 18)) each adjusted to an amount of 10 pmol were added thereto, followed by 30 PCR cycles each involving 94° C. for 20 seconds, 58° C. for 30 seconds, and 72° C. for 60 seconds, and subsequent reaction at 72° C. for 5 minutes to amplify the human or mouse FRβ gene. The PCR product of each amplified FRβ gene was ligated with a plasmid PCR2.1-TOPO (Invitrogen Corp.). Specifically, 1 μL of a NaCl solution, 1.5 μL of sterilized distilled water, and 1 μL of the vector plasmid (PCR2.1-TOPO) were added to 2.5 μl of the PCR product and incubated at room temperature for 5 minutes. A 2 μL portion thereof was added to E. coli (TOP10F). After reaction for 30 minutes in ice, the resulting bacterial cells were heat-treated at 42° C. for 30 seconds and left standing for 2 minutes in ice. 250 μL of an SOC medium was added thereto, and the cells were then cultured at 37° C. for 1 hour in a shaker. After the completion of culture, the cells were seeded over an LB medium and cultured overnight at 37° C.

For the E. coli culture, white colonies collected from the plate were added to a liquid LB medium containing ampicillin (50 μg/mL) and cultured overnight at 37° C. Plasmids were purified using Qiagen plasmid purification kit (Qiagen N.V.). The FRβ gene insert was treated with a restriction enzyme EcoRI and then developed to agarose electrophoresis to confirm the FRβ gene product of approximately 0.8 kb (782 bp). Then, the site was excised, and gene product extracts were purified using Qiagen PCR purification kit (Qiagen N.V.). Next, the purified gene product was mixed with a vector pER-BOS for expression in mammalian cells (Mizushima et al., pEF-BOS, a powerful mammalian expression vector. Nucleic Acid Res. 1990; 18 (17): 5322) treated in advance with EcoRI, and ligated therewith using T4 ligase (F. Hoffmann-La Roche Ltd.). The transfection of E. coli (TOP10F′) with the ligation product and the confirmation of the FRβ gene were performed by the same approaches as above.

After the confirmation of the FRβ gene inserted in pEF-BOS, mouse B300-19 cells were transfected with the vector containing the human FRβ gene, while rat RBL2H3 cells were transfected with the vector containing the mouse FRβ gene. Specifically, each cell line adjusted in advance to 1×10⁵ cells was transfected by the addition of 1 μg of each FRβ vector mixed with 20 μL of Lipofectamine (Gibco BRL Life Technologies, Inc.). Since the transfected mouse B300-19 cells and rat RBL2H3 cells acquire resistance to an antibiotic G418, the transfected cells of each cell line were selectively cultured in a medium containing G418 at a concentration of 1 mg/mL. The FRβ gene introduced in the transfected cells was confirmed by PCR. Specifically, cDNA was synthesized from each cell line adjusted to 1×10⁷ cells using a cDNA synthesis kit (Invitrogen Corp.). A sense primer (human rheumatoid synovium: agaaagacatgggtctggaaatggatg (SEQ ID NO: 15); or mouse liver: tctagaaagacatggcctggaaacag (SEQ ID NO: 16)) and an antisense primer (human rheumatoid synovium: gactgaactcagccaaggagccagagtt (SEQ ID NO: 17); or mouse liver: cccaacatggatcaggaact (SEQ ID NO: 18)) each adjusted to an amount of 10 pmol were added to Bioneer PCR premix (Bioneer Inc.), followed by 30 PCR cycles each involving 94° C. for 20 seconds, 58° C. for 30 seconds, and 72° C. for 60 seconds, and subsequent reaction at 72° C. for 5 minutes to amplify the human or mouse FRβ gene. After the amplification, a band of 0.8 kb exhibited by the FRβ gene was confirmed by agarose electrophoresis.

### [Preparation of Anti-Human FRβ Mouse Monoclonal Antibody and Anti-Mouse FRβ Rat Monoclonal Antibody]

The FRβ-expressing mouse B300-19 cells or rat RBL2H3 cells were separately adjusted to 1×10⁷ cells and mixed with a complete Freund's adjuvant. A Balb/C mouse (for the anti-human FRβ monoclonal antibody) or a Wistar Kyoto rat (for the anti-mouse FRβ monoclonal antibody) was immunized by the intraperitoneal administration of the antigen to three sites of the tail. This immunization was repeated twice to four times.

Each monoclonal antibody was prepared according to the method of Kohler (Kohler & Milstein, Nature (1975) 256: 495-96). Specifically, the spleen or iliac lymph node was taken out of the mouse or the rat and dissociated into single cells. The dissociated cells were fused with myeloma-derived cells (NS-1) to prepare hybridomas, which were then cultured in a HAT selective medium. Antibodies secreted into the culture supernatant were screened on the basis of reactivity with the FRβ-expressing cells.

The obtained hybridomas were adjusted to 1 cell per well of a 96-well plate and cloned by limiting dilution culture. The cloned cells were screened on the basis of reactivity with the FRβ-expressing cells.

The cloned hybridomas were adjusted to 1×10⁷ cells and intraperitoneally administered to a nude mouse to prepare ascitic fluid. The monoclonal antibodies were purified using Protein G columns (GE Healthcare Japan Corp.). The isotypes of the purified mouse and rat monoclonal antibodies were determined using Isotyping ELISA kit (BD Pharmingen, Becton Dickinson and Company). As a result, two anti-human FRp mouse monoclonal antibodies were obtained: IgG2a-type clone 36b and IgG1-type clone 94b. Meanwhile, two anti-mouse FRβ rat monoclonal antibodies were obtained: IgG2a-type CL5 and CL10. These antibodies were analyzed for their respective reactivity with the antigen by flow cytometry.

### [Determination of Heavy Chain Variable Region (VH) and Light Chain Variable Region (VL) Genes of Anti-Human FRβ Mouse Monoclonal Antibody and Anti-Mouse FRβ Rat Monoclonal Antibody]

The mouse hybridoma clones 36b and 94b were each adjusted to 1×10⁷ cells, and cDNA was synthesized therefrom using a cDNA synthesis kit (Invitrogen Corp.). The VH and VL genes of 36b and 94b were determined by PCR using Ig-Prime Kit. The PCR conditions followed the instruction manual included therein. Specifically, PCR was performed by 30 cycles each involving 94° C. for 60 seconds, 50° C. for 60 seconds, and 72° C. for 120 seconds, and subsequent reaction at 72° C. for 5 minutes to amplify the VH and VL genes. The PCR products of the amplified VH and VL genes were ligated with plasmids PCR2.1-TOPO (Invitrogen Corp.), with which E. coli (TOP10F′) was then transfected. Plasmids were purified from the transfected E. coli, and the VH and VL genes of 36b and 94b were sequenced. Their nucleotide sequences were subjected to PCR using BigDye Terminator V3.1 cycle sequencing kit (Applied Biosystems, Inc.), and the PCR products were analyzed using ABI 310 DNA sequencer.

The rat hybridoma clones CL5 and CL 10 were each adjusted to 1×10⁷ cells, and cDNA was synthesized therefrom using a cDNA synthesis kit (Invitrogen Corp.). Next, the VH and VL genes were amplified by PCR using Ig-Prime Kit and a primer (caccatggagttacttttgag (SEQ ID NO: 19)) designed for rat VH amplification. The PCR products of the amplified VH and VL genes were ligated with plasmids PCR2.1-TOPO (Invitrogen Corp.), with which E. coli (TOP10F′) was then transfected. Plasmids were purified from the transfected E. coli, and the VH and VL genes were sequenced. Their nucleotide sequences were subjected to PCR using BigDye Terminator V3.1 cycle sequencing kit (Applied Biosystems, Inc.), and the PCR products were analyzed using ABI 310 DNA sequencer.

### Example 2

### Accumulation of Anti-Mouse FRβ Rat Monoclonal Antibody to Arteriosclerosis Immunohistological Detection of FRβ-Expressing Macrophage in Arteriosclerosis Mouse Model

### [Immunohistochemical Staining of Mouse Arteriosclerosis Tissue]

### (Preparation of Arteriosclerosis Model)

In this Example, five 35-week-old apolipoprotein E (ApoE)-knockout mice (ApoE−/−) (The Jackson Laboratory) were used. These five mice with arteriosclerosis were evaluated for macrophages in their aortic arteriosclerotic lesions by immunohistochemical staining.

Specifically, after inhalation anesthesia with diethyl ether, each mouse with arteriosclerosis was euthanized by the intraperitoneal administration of 150 μg of a Nembutal stock solution diluted 10-fold with saline. The mouse was disinfected with 70% ethanol, and its chest was then opened by median sternotomy. The heart and aorta were exposed, and a mass from the heart to the ascending aorta was isolated.

The isolated tissue was embedded in an OCT compound to prepare a frozen tissue block. The frozen block was sliced into 5 μm using a cryostat to prepare consecutive frozen tissue specimens. The specimens thus prepared were fully dried at room temperature and then fixed in acetone. The acetone-fixed tissue specimens were washed with a phosphate-buffered saline (PBS; pH 7.3, 0.15 M NaCl) to remove the compound. Then, endogenous peroxidase was digested using PBS containing 0.15% hydrogen peroxide, followed by further washing with PBS. After the washing, PBS containing 3% casein was added dropwise onto the tissue, which was then cultured at room temperature for 30 minutes. After the culture, each anti-mouse FRβ mouse monoclonal antibody diluted 200-fold or an anti-mouse macrophage marker monoclonal antibody (CD68, AbD SeroTec, Bio-Rad Laboratories, Inc.) diluted 400-fold was added dropwise thereto and left standing at room temperature for 60 minutes. After washing with PBS, a secondary antibody was added dropwise thereto and reacted at room temperature for 30 minutes using Histofine MAX-PO (Nichirei Corp.). After the completion of reaction, the tissue was washed with an excess of PBS, and FRβ- and CD68-positive cells were detected using NOVA RED (Vector Laboratories, Inc.). This section was washed with PBS and nuclearly stained for 1 minute with a Mayer's hematoxylin solution (Muto Pure Chemicals Co., Ltd.). Then, the section was washed with running water for approximately 5 minutes and included.

### [Results]

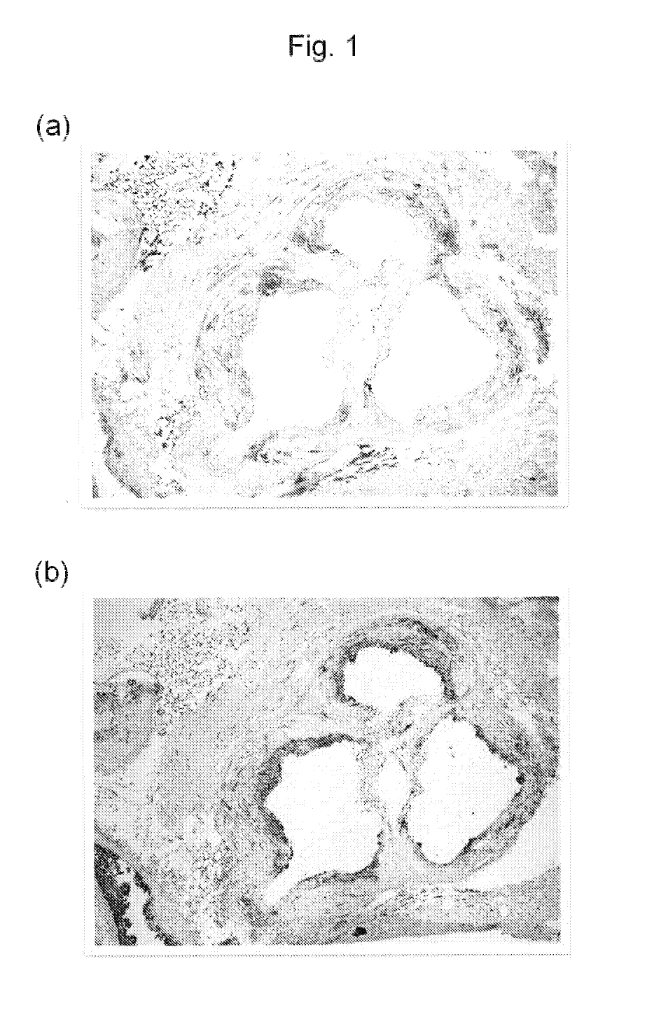

The staining results are shown in FIG. 1. FIG. 1(a) shows the results of staining with the anti-mouse FRβ mouse monoclonal antibody. FIG. 1(b) shows the results of staining with the anti-mouse macrophage marker monoclonal antibody. As is evident from FIGS. 1(a) and 1(b), the localization of the CD68-targeting macrophage marker and the localization of FRβ mostly overlapped with each other, demonstrating that many of activated macrophages present in the arteriosclerotic lesion of the arteriosclerosis mouse model highly express FRβ. This result suggested the possibility that the selective removal of such FRβ-expressing macrophages is effective for the treatment of arteriosclerosis, particularly, the treatment of an active and unstable arteriosclerotic lesion.

### Example 3

### Production of Recombinant Immunotoxin

### [Introduction of Cysteine Mutation to Heavy Chain Variable Region (VH) of Immunoglobulin]

Primers (sense primer: cagaggcctgaacattgtctggagtggattggaag (SEQ ID NO: 20) and antisense primer: cttccaatccactccagacactgttcaggcctctg (SEQ ID NO: 21)) designed to mutate glycine (nucleotide sequence: ggc) at amino acid 63 to cysteine (nucleotide sequence: tgt) in the immunoglobulin heavy chain variable region (VH) of the anti-human FRβ mouse monoclonal antibody 94b were prepared and used in the mutagenesis treatment of the 94b VH gene-containing plasmid pCR2.1-TOPO 94bVH obtained in Example 1 using Quick change site-directed mutagenesis kit (Stratagene Corp.). This PCR was performed using a reaction solution by 12 continuous cycles each involving 95° C. for 30 seconds, 55° C. for 60 seconds, and 68° C. for 4 minutes. Cysteine was introduced to the immunoglobulin heavy chain variable region (VH) of the anti-mouse FRβ rat monoclonal antibody CL10 in the same way as above using designed primers (sense primer: gtccgccaggctccaacgaagtgtctggagtgggtcgc (SEQ ID NO: 22) and antisense primer: gcgacccactccagacacttcgttggagcctggcggac (SEQ ID NO: 23)).

Next, E. coli XL1-Blue was transfected with each DNA thus reacted and selectively cultured in an LB medium containing 0.1 mg/mL ampicillin. The plasmid of the selected transformant was purified using QIAprep spin Miniprep KIT (Qiagen N.V.). Its nucleotide sequence was further determined using Big Dye Terminator v3.1 cycle sequencing kit (Applied Biosystems, Inc.) and ABI310 sequencer to confirm the successful gene mutation of glycine to cysteine (nucleotide sequence: tgt) at amino acid 63.

[Insertion of Mutated VH Gene to pRK79PE38 Vector]

Next, the mutated VH genes of 94bVH and CL10VH were each inserted to a PE38 gene-containing pRK79 vector pRK79PE38 by the following method:

Annealing primers taagaaggagatatacatatggaggttcagctgcagcagtc (SEQ ID NO: 24) and gccctcgggacctccggaagcttttgaggagactgtgagagtgg (SEQ ID NO: 25) were designed for the 5′ and 3′ ends of the mutated 94bVH gene. Annealing primers atacatatggaggtgcagctggtggagtctggg (SEQ ID NO: 26) and tccggaagcttttgaggagacagtgactgaagc (SEQ ID NO: 27) were designed for the 5′ and 3′ ends of the mutated CL1 OVH gene. One of the annealing primers in each set has recognition site for a restriction enzyme NdeI. Cloning at this site permits protein expression with atg as a start codon. Also, a HindIII recognition site is inserted in the other annealing primer. Cloning at this site permits expression of a fusion protein from the VH gene bound with the PE38 gene.

The mutated pCR2.1-TOPO-94bVH and pCR2.1-TOPO-CL10VH plasmids were subjected to PCR using these primer sets and Pfu DNA polymerase (Stratagene Corp.). This reaction was performed by 30 PCR cycles each involving 94° C. for 20 seconds, 55° C. for 30 seconds, and 72° C. for 60 seconds, and subsequent reaction at 72° C. for 5 minutes. Next, each PCR product was purified, and the purified product was reacted by the addition of restriction enzymes NdeI (New England Biolabs Inc.) and HindIII (New England Biolabs Inc.) and then developed to electrophoresis. DNA with the size of interest was collected from the gel using QIAquick gel extraction kit (Qiagen N.V.). pRK79PE38 treated with the same restriction enzymes as in the restriction enzyme-treated mutated VH gene was added to the collected DNA. Then, the ligation reaction between the VH gene and pRK79PE38 was performed using Ligation High (Toyobo Co., Ltd.). After the completion of ligation reaction, E. coli TOP10F′ (Invitrogen Corp.) was transfected with the ligation product, and a transformant was selected in an LB medium containing 0.1 mg/mL ampicillin. The plasmid pRK79-VHPE of the selected transformant was purified using QIAprep spin Miniprep KIT (Qiagen N.V.). Its nucleotide sequence was further determined using Big Dye Terminator v3.1 cycle sequencing kit (Applied Biosystems, Inc.) and ABI310 sequencer to confirm the successful ligation of the nucleotide sequence of the mutated VH gene with the PE38 nucleotide sequence of the pRK79 vector.

### [Introduction of Cysteine Mutation to Light Chain Variable Region of Immunoglobulin]

Primers designed to mutate amino acid 125 to cysteine (nucleotide sequence: tgt) in the immunoglobulin light chain variable region (VL) of the anti-human FRβ mouse monoclonal antibody 94b were prepared (sense primer: taagaaggagatatacatatggacattgtgatgtcacaatc (SEQ ID NO: 28); since this primer contains bases catatg cleavable with a restriction enzyme NdeI, cloning at this site permits protein expression with atg as a start codon) and (antisense primer: gctttgttagcagccgaattcctatttgatttccagcttggtgccacaaccgaacgt (SEQ ID NO: 29); since this primer was designed to mutate amino acid 125 to cysteine (tgt) and position a stop codon tag followed by bases gaattc cleavable with a restriction enzyme EcoRI). Likewise, primers designed to mutate amino acid 125 to cysteine (nucleotide sequence: tgt) in the immunoglobulin light chain variable region (VL) of the anti-mouse FRβ rat monoclonal antibody CL10 were prepared (atacatatggacattgtgatgcccaatctccatcc (SEQ ID NO: 30) and gctttgttagcagccgaattcctatttgatttccagcttggtgccacaaccgaacgt (SEQ ID NO: 31)).

The pCR2.1-TOPO-94bVL plasmid was subjected to PCR using these primer sets and Pfu DNA polymerase (Stratagene Corp.). This reaction was performed by 30 PCR cycles each involving 94° C. for 20 seconds, 55° C. for 30 seconds, and 72° C. for 60 seconds, and subsequent reaction at 72° C. for 5 minutes. Next, each PCR product was purified, and the purified product was reacted by the addition of restriction enzymes NdeI (New England Biolabs Inc.) and EcoRI (New England Biolabs Inc.) and then developed to electrophoresis. DNA with the size of interest was collected from the gel using QIAquick gel extraction kit (Qiagen N.V.). pRK79PE38 treated with the same restriction enzymes as in the restriction enzyme-treated mutated VL gene was added to the collected DNA. Then, the ligation reaction between the VH gene and pRK79PE38 was performed using Ligation High (Toyobo Co., Ltd.). After the completion of ligation reaction, E. coli TOP10F′ (Invitrogen Corp.) was transfected with the ligation product, and a transformant was selected in an LB medium containing 0.1 mg/mL ampicillin. The plasmid pRK79-VLPE of the selected transformant was purified using QIAprep spin Miniprep KIT (Qiagen N.V.). Its nucleotide sequence was further determined using Big Dye Terminator v3.1 cycle sequencing kit (Applied Biosystems, Inc.) and ABI310 sequencer to confirm the successful gene mutation of amino acid 125 to cysteine in mutated VL and position of the stop codon tag.

### [Preparation of Recombinant Protein Inclusion Body]

The plasmids pRK79-94bVHPE and pRK79-CL10VHPE each having the mutated VH gene insert and the plasmids pRK79-VL94b and pRK79-VLCL10 each having the mutated VL gene insert were separately adjusted to 50 ng, with which E. coli BL21(DE3) for protein expression was then transfected. The transfected E. coli cells were screened by culture at 37° C. for 15 to 18 hours in an LB medium containing 0.1 mg/mL ampicillin.

After the completion of selection, the E. coli was cultured in 1000 mL of a Super Broth medium under conditions of 37° C. and cultured until the absorbance of visible light reached 1.0 to 1.5 at 600 nm. After the culture, isopropyl-beta-D-thiogalactopyranoside (IPTG) was added at a final concentration of 1 mM to the medium, and the E. coli was further cultured at 37° C. for 90 minutes. After the completion of culture, the E. coli was collected by centrifugation and then suspended to 200 mL using a 50 mM tris buffer solution (pH 7.4; containing 20 mM EDTA). After the completion of suspension, egg-white lysozyme was added thereto at a final concentration of 0.2 mg/mL and reacted at room temperature for 1 hour to disrupt the E. coli. The E. coli thus disrupted was centrifuged at 20,000×g, and the precipitate was collected. The precipitate was further suspended to 200 mL in a 50 mM tris buffer solution (pH 7.4; containing 2.5% Triton X-100, 0.5 M NaCl, and 20 mM EDTA). Egg-white lysozyme was added thereto at a final concentration of 0.2 mg/mL and reacted at room temperature for 1 hour. After the completion of reaction, the reaction product was centrifuged at 20,000×g, and the precipitate was collected. The precipitate was further suspended to 200 mL in a 50 mM tris buffer solution (pH 7.4; containing 2.5% Triton X-100, 0.5 M NaCl, and 20 mM EDTA), sufficiently mixed, and then centrifuged at 20,000×g, and the precipitate was collected. This operation was repeated five times, and the resulting precipitate was used as a recombinant immunotoxin inclusion body, which was further dissolved in a 0.1 M tris buffer solution (pH 8.0; containing 6 M guanidine hydrochloride and 1 mM EDTA) to adjust the final concentration to 10 mg/mL.

### [Preparation of Recombinant Double-Chain Fv Anti-FRβ Immunotoxin]

The 94b-VHPE and 94b-VL or CL10-VHPE and CL10-VL prepared above were mixed to prepare recombinant double-chain Fv anti-FRβ immunotoxins.

First, 0.5 mL of each VHPE and 0.25 mL of its corresponding VL were mixed and reduced at room temperature for 4 hours by the addition of dithiothreitol (DTT) at a final concentration of 10 mg/mL. The mixture thus treated was dissolved in 75 mL of a 0.1 M tris buffer solution (pH 8.0; containing 0.5 M arginine, 0.9 mM oxidized glutathione, and 2 mM EDTA). This solution was left at 10° C. for 40 hours to link VH to VL. After the completion of linking, the linking product was concentrated into 5 mL using a centrifugal concentrator with a molecular weight cutoff of 10,000 (Centricon 10, Amicon, Millipore Corp.) and further diluted with 50 mL of a tris buffer solution (pH 7.4; containing 0.1 M urea and 1 mM EDTA). This diluted solution was used as a starting material for recombinant immunotoxin purification.

Next, the starting material was adsorbed at a flow rate of 30 mL/hour onto an ion-exchange column Hi-Trap Q (GE Healthcare Japan Corp.) equilibrated with a tris buffer solution (pH 7.4; containing 1 mM EDTA) and then washed with a tris buffer solution (pH 7.4; containing 1 mM EDTA). After the washing, each adsorbed recombinant-type immunotoxin was eluted with a tris buffer solution (pH 7.4; containing 0.3 M NaCl and 1 mM EDTA). The eluted sample was dialyzed against a tris buffer solution (pH 7.4; containing 1 mM EDTA) and then further purified using an ion-exchange column POROS HQ (POROS, Applied Biosystems, Inc.). Specifically, the dialyzed purified substance was adsorbed onto the column at a flow rate of 10 mL/minute and washed with a tris buffer solution (pH 7.4; containing 1 mM EDTA), followed by elution of the recombinant-type immunotoxin using the buffer solution with NaCl gradients set to from 0 M to 1.0 M. The purified recombinant-type immunotoxin was finally adjusted by TSK300SW (Tosoh Corp.) gel filtration chromatography. First, endotoxins in the TSK300SW column were washed off for 48 hours using 75% ethanol for disinfection. Next, the TSK300SW column was washed with Japanese Pharmacopoeia distilled water for injection and then equilibrated with Japanese Pharmacopoeia saline. After the completion of equilibration, the recombinant-type immunotoxin was administered to the column, and an eluate from the column was recovered at a flow rate of 0.25 mL/minute. The eluate thus recovered was treated with a 0.22-μm sterilization filter and stored at −80° C. after purity confirmation by SDS-PAGE.

### [Purity Assay by SDS-PAGE]

SDS-PAGE (electrophoresis on a polyacrylamide gel containing sodium dodecyl sulfate) employed a 12% polyacrylamide slab gel containing 0.1% sodium dodecyl sulfate (SDS) and employed an aqueous solution containing 0.1% SDS, 130 mM glycine, and 25 mM tris (the concentrations were indicated by final concentrations) as a mobile phase. Each sample was adjusted with a 100 mM tris buffer solution (pH 6.5) containing 0.1% (final concentration) SDS and boiled for 5 minutes. After the completion of boiling, the sample was administered to the slab gel and developed by electrophoresis at a constant current of 30 mA. The recombinant-type immunotoxin thus electrophoresed was stained with a 0.05% Coomassie Brilliant Blue R solution (Nacalai Tesque, Inc.).

### Example 4

### Verification of Therapeutic Effects of Anti-Human FRβ Antibody and Recombinant Immunotoxin on Arteriosclerosis

The animal models used were 15-week-old apolipoprotein E (ApoE)-knockout mice (ApoE−/−). This Example employed the recombinant immunotoxin prepared in Example 3 (immunotoxin-administered group), the antibody (anti-human FRβ mouse monoclonal antibody) prepared in Example 1 (antibody-administered group), and a placebo lacking binding activity against FRβ (fusion protein of PE38 and VH of the anti-human FRβ mouse monoclonal antibody 94b) (placebo-administered group) and saline (control group) as controls. Five mouse individuals were assigned to each of the immunotoxin-administered group, the antibody-administered group, the placebo-administered group, and the control group. The recombinant immunotoxin or the placebo corresponding to 2.5 μg diluted with 0.1 ml of saline was intravenously injected to the tail vein of each mouse a total of five times at three-day intervals during a period of 15 week olds to 17 week olds. 0.3 mg of the antibody diluted with 0.1 ml of saline was intravenously injected to the tail vein of each mouse in the antibody-administered group. 0.1 ml of saline was intravenously injected to the tail vein of each mouse in the control group.

### [Immunohistochemical Analysis]

28 days after the administration, each mouse was euthanized, and its heart and ascending aorta were resected to prepare frozen tissue specimens. Immunohistochemical analysis was conducted in the same way as in Example 2 above.

An arteriosclerotic lesion in the aortic sinus was examined at 15 positions spaced 50 μm from the initial site where the arteriosclerotic lesion of the aortic root appeared, and then stained with Oil Red O. In order to quantify the lesion in the aorta, each image under a microscope was digitized and analyzed using Scion Image software. Oil Red O-positive regions were analyzed by comparison with the vascular wall region of a cross section of the whole sample. An average value of 15 positions for each animal was determined.

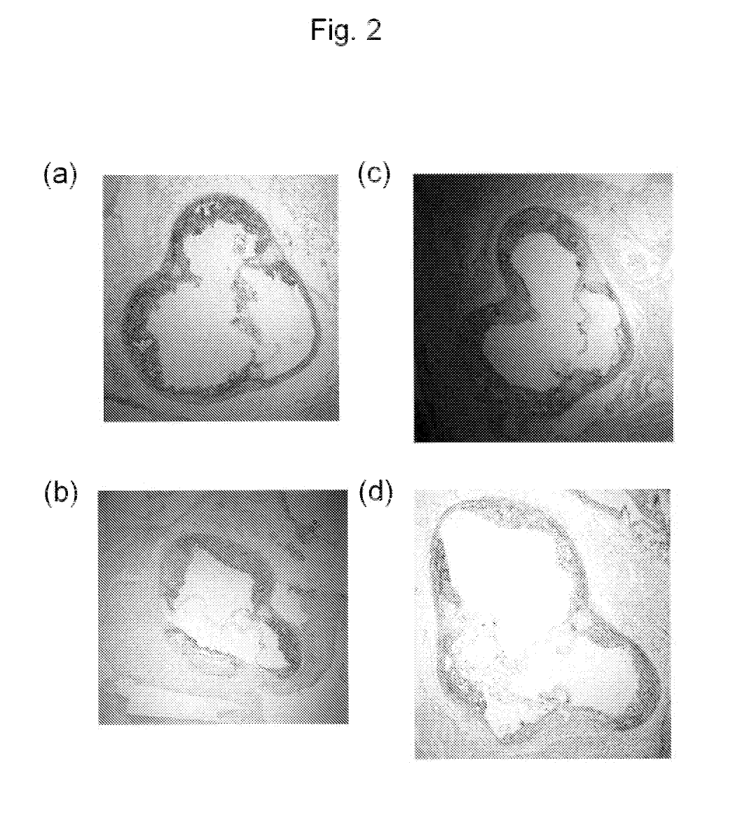

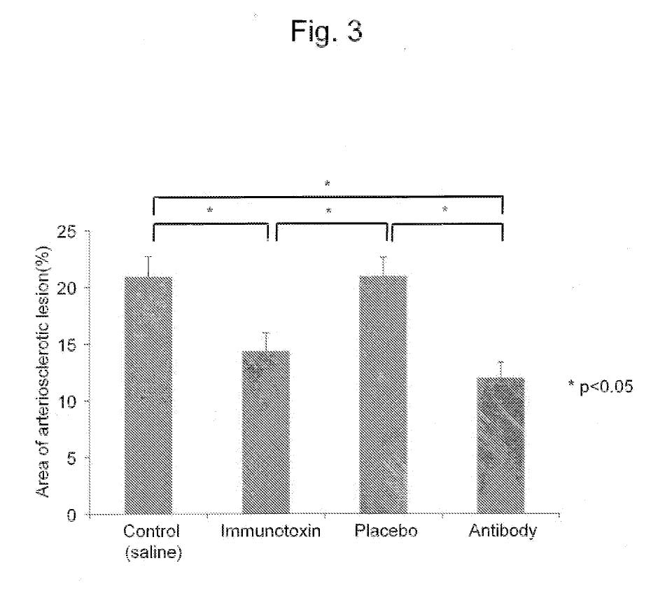

FIG. 2 shows one example of the results of Oil Red O staining of the arteriosclerotic lesions in the immunotoxin-administered group, the antibody-administered group, the placebo-administered group, and the control group. In this context, FIG. 2(a) shows the results of Oil Red O staining of the arteriosclerotic lesion in the immunotoxin-administered group. FIG. 2(b) shows the results of Oil Red O staining of the arteriosclerotic lesion in the control group. FIG. 2(c) shows the results of Oil Red O staining of the arteriosclerotic lesion in the placebo-administered group. FIG. 2(d) shows the results of Oil Red O staining of the arteriosclerotic lesion in the antibody-administered group. Also, the results of quantifying the lesion in the aorta are shown in FIG. 3.

As shown in FIGS. 2 and 3, the significant involution of the arteriosclerotic lesion was confirmed in the immunotoxin-administered group and the antibody-administered group. Particularly, in this Example, arteriosclerosis was suppressed, as shown in FIG. 3, by 31% in the immunotoxin-administered group and by 43% in the antibody-administered group. As described above, the immunotoxin and the antibody reduced the size of the unstable and active arteriosclerotic lesion rich in lipid components indicated by the Oil Red O staining, suggesting that the immunotoxin and the antibody can each be used in the treatment of the active and unstable arteriosclerotic lesion.

### [Measurement of the Number of Peripheral Blood Monocytes]

Total blood was collected using an injection needle coated with 1000 U/ml heparin from the heart of each mouse in the immunotoxin-administered group, the antibody-administered group, the placebo-administered group, and the control group euthanized in the immunohistochemical analysis described above. The number of monocytes was measured using a blood cell counter.

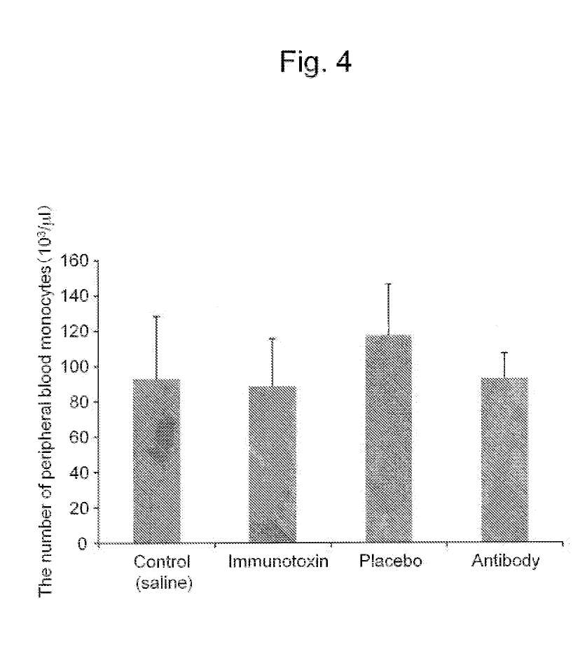

The measurement results are shown in FIG. 4. As shown in FIG. 4, no significant difference in the number of peripheral blood monocytes was confirmed among the immunotoxin-administered group, the antibody-administered group, the placebo-administered group, and the control group.

### [Measurement of Blood Lipid Level]

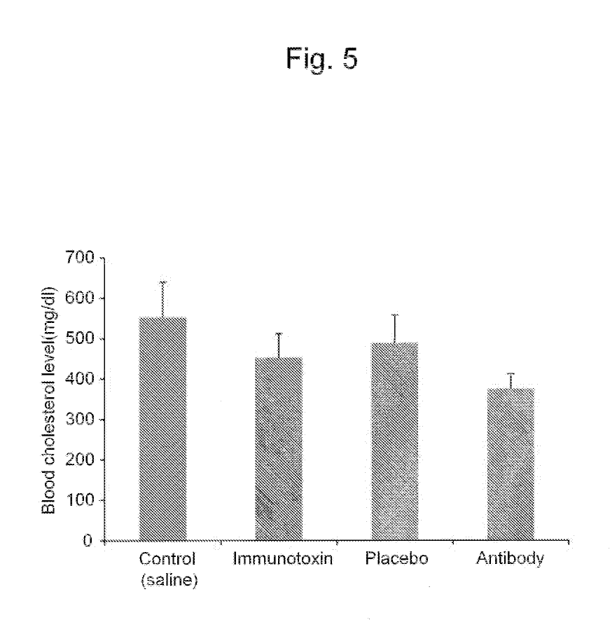

Also, total cholesterol levels in blood at the time of food satiation were measured using T-CHO Kainos (Kainos Laboratories, Inc.). The measurement results are shown in FIG. 5. As shown in FIG. 5, no significant difference in total cholesterol levels in blood was confirmed among the immunotoxin-administered group, the antibody-administered group, the placebo-administered group, and the control group.

### [Measurement of the Number of FRβ-Expressing Macrophages in Arteriosclerotic Lesion]

In addition, frozen tissue specimens were prepared from the heart and ascending aorta of each mouse obtained 28 days after the completion of administration to the immunotoxin-administered group, the antibody-administered group, the placebo-administered group, and the control group, and then immunohistochemically analyzed. Each image under a microscope was digitized (DS-Fil, Nikon Corp., Tokyo, Japan) and analyzed using imaging software (NIS-Elements, Nikon Corp.). The number of FRβ-expressing cells in the arteriosclerotic lesion of the aortic root was calculated and compared among the immunotoxin group, the antibody-administered group, the placebo-administered group, and the control group.

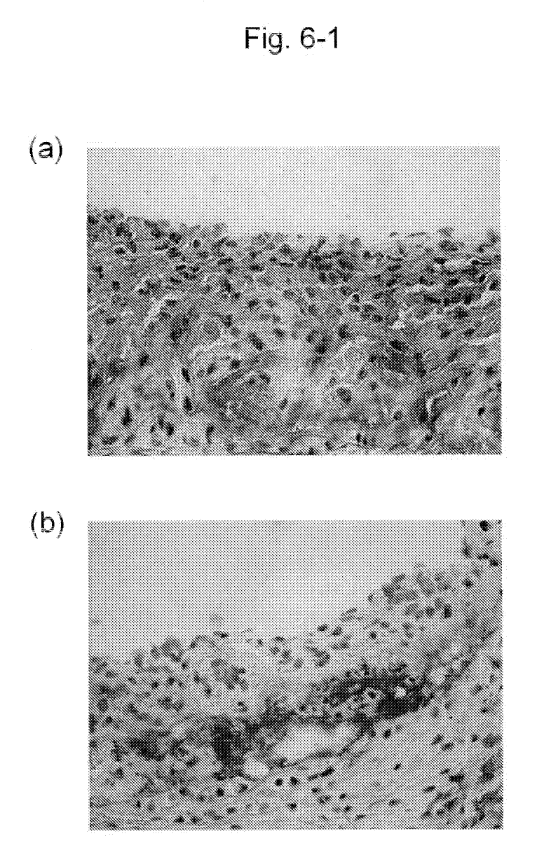

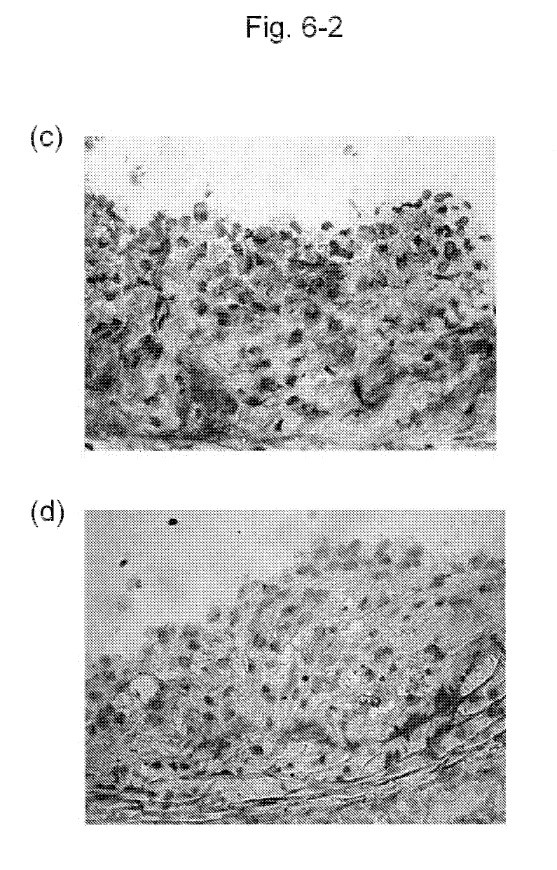

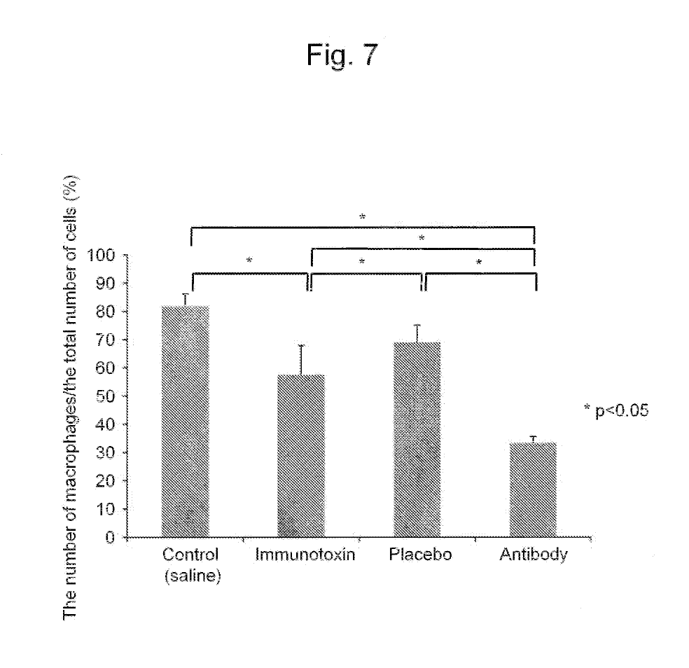

FIGS. 6-1 and 6-2 show the immunostaining results of the immunotoxin-administered group, the antibody-administered group, the placebo-administered group, and the control group. In this context, FIG. 6-1(a) shows the results of immunostaining of the arteriosclerotic lesion in the control group. FIG. 6-1(b) shows the results of immunostaining of the arteriosclerotic lesion in the immunotoxin-administered group. FIG. 6-2(c) shows the results of immunostaining of the arteriosclerotic lesion in the placebo-administered group. FIG. 6-2(d) shows the results of immunostaining of the arteriosclerotic lesion in the antibody-administered group. FIG. 7 shows the results of comparing the counted number of macrophages in the arteriosclerotic lesion among the immunotoxin-administered group, the antibody-administered group, the placebo-administered group, and the control group.

As shown in FIGS. 6-1, 6-2, and 7, the FRβ-expressing macrophages were removed in the immunotoxin-administered group and the antibody-administered group, further demonstrating that the arteriosclerotic lesion was suppressed.

The results described above demonstrated that the recombinant immunotoxin prepared in Example 3 and the antibody prepared in Example 1 have the pharmacological effect of bringing about cell death to the activated macrophage present in the arteriosclerotic lesion, resulting in the involution of the arteriosclerotic lesion, particularly, the active and unstable arteriosclerotic lesion. Furthermore, these results showed that the selective removal of the FRβ-expressing macrophage present in the arteriosclerotic lesion is effective for the treatment of arteriosclerosis, particularly, the treatment of the active and unstable arteriosclerotic lesion, and the treatment of arteriosclerotic disease.

The FRβ-expressing macrophage was also shown to be present in the unstable and active arteriosclerotic lesion rich in lipid components indicated by Oil Red O staining. This result demonstrated that the active arteriosclerotic lesion having the unstable plaque can be detected with FRβ as an index. Specifically, the active arteriosclerotic lesion having the unstable plaque can be identified using the anti-FRp antibody such as the anti-mouse FRβ rat monoclonal antibody as prepared in Example 2.

### Example 5

### Immunohistological Staining of Human Carotid Artery Tissue with Anti-Human FRp Mouse Monoclonal Antibody

### [Paraffin-Embedded Specimen]

In this Example, paraffin-embedded specimens of human carotid artery tissues were prepared as described below. Tissues obtained by carotid endarterectomy from a patient with high-grade internal carotid artery stenosis were fixed in formalin. Then, the tissues were embedded in paraffin, and 5-μm paraffin sections were then prepared.

### [Immunohistochemical Analysis]

The specimens were deparaffinized and then subjected to antigen retrieval by autoclaving using Diva Decloaker. The tissue was incubated for 10 minutes in a 0.3% aqueous hydrogen peroxide solution to block endogenous peroxidase reaction. Then, the tissue was incubated with 10% normal goat serum at room temperature for 30 minutes.

The anti-human FRβ mouse monoclonal antibody prepared in Example 1 was reacted therewith overnight at 4° C. After washing with PBS, the tissue was incubated with Simple Stain Max-PO as a secondary antibody at room temperature for 30 minutes. After washing with PBS, a color was developed using Nova Red, and the tissue was included in Vectamount.

### [Results]

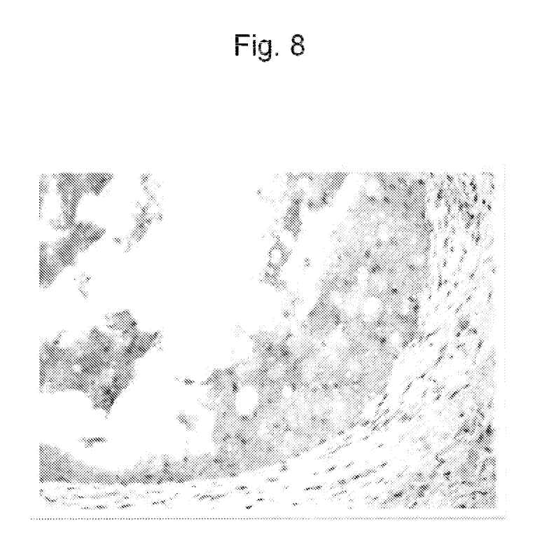

The staining results are shown in FIG. 8. As is evident from FIG. 8, many of activated macrophages present in the arteriosclerotic lesion of the human carotid artery tissue were also shown to highly express FRβ. This result and the result of Example 4 strongly suggested that the recombinant immunotoxin prepared in Example 3 and the anti-human FRβ antibody prepared in Example 1 have the pharmacological effect, on humans, of bringing about cell death to the activated macrophage present in the human arteriosclerotic lesion, resulting in the involution of the arteriosclerotic lesion, particularly, the active and unstable arteriosclerotic lesion.

### Example 6

### Detection of Arteriosclerotic Lesion by Molecular Imaging Using Anti-Human FRβ Mouse Monoclonal Antibody

### [Molecular Imaging]

In this Example, 35-week-old apolipoprotein E (ApoE)-knockout mice (ApoE−/−) (The Jackson Laboratory) were used. The anti-human FRβ mouse monoclonal antibody prepared in Example 1 was labeled with Alexa Fluor 488 (manufactured by Invitrogen Corp.) and administered at a dose of 100 μg/mouse by intravenous injection from the tail vein of each mouse.

Two hours after the administration by intravenous injection, each mouse was slaughtered by the same approach as in Example 2, and the whole aorta was isolated according to a standard method. The isolated aorta was excited at 488 nm using Maestro™ in-vivo imaging system (manufactured by Cambridge Research & Instrumentation, Inc.) and photographed at 520 nm.

### [Results]

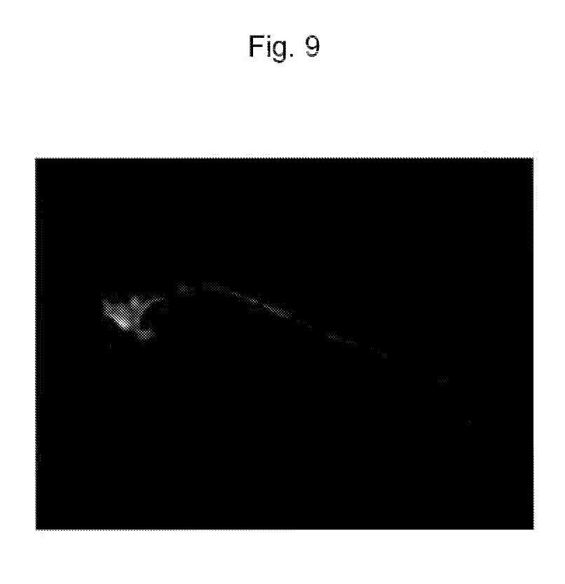

The imaging results are shown in FIG. 9. As shown in FIG. 9, molecular imaging using a fluorescent label was shown to be able to detect the arteriosclerotic lesion (arteriosclerotic lesion site). This result suggested that a site at a high risk of thrombus formation accompanying the rupture of the unstable and active arteriosclerotic lesion (unstable plaque) rich in lipid components can be image-diagnosed.

### INDUSTRIAL APPLICABILITY

The therapeutic agent for arteriosclerosis or arteriosclerotic disease of the present invention can selectively induce the cell death or cell damage of an activated macrophage present in an arteriosclerotic lesion and thereby cause the involution of the arteriosclerotic lesion. Also, the diagnostic agent for arteriosclerosis or arteriosclerotic disease of the present invention can detect an activated macrophage present in an arteriosclerotic lesion and thereby identify the arteriosclerotic lesion.

All publications, patents, and patent applications cited herein are incorporated herein by reference in their entirety.

1.-15. (canceled)

16. A therapeutic agent for arteriosclerosis or arteriosclerotic disease, comprising a complex comprising an antibody specifically binding to folate receptor β (FRβ) and a cytotoxin or a cytotoxic agent conjugated with the antibody, or the antibody as an active ingredient.

17. The therapeutic agent according to claim 16, wherein the arteriosclerosis is atherosclerosis.

18. The therapeutic agent according to claim 16, wherein the arteriosclerotic disease is one selected from the group consisting of cerebral infarction, cerebral hemorrhage, ischemic heart disease, aortic aneurysm, aortic dissection, nephrosclerosis, renal failure, and arteriosclerosis obliterans.

19. The therapeutic agent according to claim 16, wherein the complex is a recombinant immunotoxin.

20. The therapeutic agent according to claim 16, wherein the antibody does not bind to folate receptor a.

21. The therapeutic agent according to claim 16, wherein the antibody is a chimeric antibody, a humanized antibody, or a human antibody.

22. The therapeutic agent according to claim 16, wherein the antibody comprises an amino acid sequence comprising at least one complementarity determining region (CDR) in the respective amino acid sequences of any heavy chain (H chain) variable region and/or any light chain (L chain) variable region of an anti-human folate receptor β mouse monoclonal antibody or an anti-mouse folate receptor β rat monoclonal antibody.

23. The therapeutic agent according to claim 16, wherein the cytotoxin is selected from the group consisting of Pseudomonas aeruginosa exotoxin, ricin A chain, deglycosylated ricin A chain, ribosome inactivating protein, alpha-sarcin, gelonin, aspergillin, restrictocin, ribonuclease, epipodophyllotoxin, and diphtheria toxin.

24. A diagnostic agent for arteriosclerosis or arteriosclerotic disease, comprising an antibody specifically binding to folate receptor β (FRβ) as an active ingredient.

25. The diagnostic agent according to claim 24, wherein atherosclerosis is diagnosed as the arteriosclerosis.

26. The diagnostic agent according to claim 24, wherein one selected from the group consisting of cerebral infarction, cerebral hemorrhage, ischemic heart disease, aortic aneurysm, aortic dissection, nephrosclerosis, renal failure, and arteriosclerosis obliterans is diagnosed as the arteriosclerotic disease.

27. The diagnostic agent according to claim 24, wherein the antibody does not bind to folate receptor α.

28. The diagnostic agent according to claim 24, wherein the antibody is a chimeric antibody, a humanized antibody, or a human antibody.

29. The diagnostic agent according to claim 24, wherein the antibody comprises an amino acid sequence comprising at least one complementarity determining region (CDR) in the respective amino acid sequences of any heavy chain (H chain) variable region and/or any light chain (L chain) variable region of an anti-human folate receptor β mouse monoclonal antibody or an anti-mouse folate receptor β rat monoclonal antibody.

30. The diagnostic agent according to claim 24, wherein the diagnostic agent identifies an unstable plaque site in the arteriosclerotic lesion.


SEQUENCE LISTING (31 sequences)

SEQ ID NO: 1 - protein, 208 aa, Homo sapiens
DRTDLLNVCMDAKHHKTKPGPEDKLHDQCSPWKKNACCTASTSQELHKDTSRLYNFNWDHCGKMEPACKRHFIQDTCLYECSPNLGPWIQQVNQTWRKERFLDVPLCKEDCQRWWEDCHTSHTCKSNWHRGWDWTSGVNKCPAGALCRTFESYFPTPAALCEGLWSHSYKVSNYSRGSGRCIQMWFDSAQGNPNEEVARFYAAAMHVN

SEQ ID NO: 2 - protein, 206 aa, Mus musculus
DRTDLLNVCMDAKHHKTKPGPEDKLHDQCSPWKKNACCSVNTSQELHKADSRLYFNWDHCGKMEPACKSHFIQDSCLYECSPNLGPWIQQVDQSWRKERFLDVPLCKEDCHQWWEACRTSFTCKRDWHKGWDWSSGINKCPNTAPCHTFEYYFPTPASLCEGLWSHSYKVSNYSRGSGRCIQMWFDSTQGNPNEDVVKFYASFMTS

SEQ ID NO: 3 - DNA, 375 bp, Mus musculus
caggttcagctgcagcagtctggagctgagctgatgaagcctggggcctcactgaagatatcctgcaaggctactggctacacattcagtagctactggatagagtgggtaaagcagaggcctggacatggccttcagtggattggagaaattttgcctggaagtggtagttctaactacaatgagaagttcaagggcaaggccacattcactgcagatacatcctccaacacagcctacatgcaactcagcagcctgacatctgaggactctgccgtctattactgtgcaagacggggcagctcgggctcttactttgctatggactactggggtcaaggaacctcagtcaccgtctcctcataggaattcggc

SEQ ID NO: 4 - DNA, 328 bp, Mus musculus
gacatccagatgacacaatcttcatcctccttttctgtatctctaggagacagagtcaccattacttgcaaggcaagtgaggacatatataatcggttagcctggtatcagcagaaaccaggaaatgctcctaggctcttaatatctggtgcaaccagtttggaaactggggttcctttaagattcagtggcagtggatctggaaaggattacactctcagcattaccagtcttcagactgaagatattgctacttattactgtcaacagtattggagtactccgtggacgttcggtggaggcaccaagctggaaatcaaaaaagctt

SEQ ID NO: 5 - DNA, 447 bp, Mus musculus
atggaatggacctgggtcattctcttcctgatggcagtggttacaggggtcaattcagaggttcagctgcagcagtctggggcagaacttgtgaagccaggggcctcagtcaggttgtcctgcacagcttctggcttcaacattaaagacacctacatgtactgggtgaaacagaggcctgaacagggcctggagtggattggaaggcttgatcctgcgaatggtaatactagatatgacccgaagttccagggcaaggccactataacatcagacacatcctccaacacagcctacctgcaactcagcagcctgacatctgaggacactgccgtctattactgtgcgggggactacccgataattgactactggggccaaggcaccactctcacagtctcctcagccaaaacgacacccccacccgtctatccactggcccctgga

SEQ ID NO: 6 - DNA, 450 bp, Mus musculus
atggttctcatcttgctgctgctatgggtatctggtgagaaatttaaaggtacctgtggggacattgtgatgtcacaatctccatcctccctggctgtgtcagcaggagagaaggtcactatgagctgcaaatccagtcagagtctgctcaacagtagaacccgaaagacctacttggcttggtatcagcagaaaccagggcagtctcctaaactgctgatctactgggcatccactagggaatctggggtccctgatcgcttcacaggcagtggatctgggacagagttcactctcaccatcagcagtgtgcaggctgaagacctggcagtttattactgcaagcaatcttataatctgtggacgttcggtggaggcaccaagctggaaatcaaacgggctgatgctgcaccaactgtatccatcttcccgccatccagtaagctggga

SEQ ID NO: 7 - DNA, 539 bp, Rattus norvegicus
atgaagttcgggttcagcttggttttccttgtccttttcataaaaggtgtccagtgtgaggtgcagctggtggagtctgggggaggcttagtgcagcctggaaggtccctgaaactctcctgtgcagcctcaggattcactttcagtaactatgacatggcctgggtccgccaggctccaacgaagggtctggagtgggtcgcatccattagtcctagtggtggtatcccttactatcgagactccgtgaagggccgattcactgtctccagagataatgcaaaaagcaccctatacctgcaaatggacagtctgaggtctgaggacacggccacttattactgtgcaagacatgggcctacggaggcccccgatgttatggatgcctggggtcaaggagcttcagtcactgtctcctcagctgaaacaacagccccatctgtctatccactggctcctggaactgctctcaaaagtaactccatggtgaaatgggatccgtctagatcgacgggcaacagcaactggcgaacaggcaa

SEQ ID NO: 8 - DNA, 671 bp, Rattus norvegicus
atggtcctcatatccttgctgttctggatttctgggacattgtgacccaatctccatcctctctggctgtgtcagcaggagagacggtcactataaactgcaagtccagtcagagtcttttatacagtggaaaccaaaagaactacttggcctggtaccagcagaaaccagggcagtctcctaaactgctgatctactgggcatctactaggcaatctggtgtccctgatcgcttcataggcagtggatctgggacagacttcactctgaccatcagcagtgtgcaggcagaagatctggcaatttattactgtcagcagtattatgatactccgctcacgttcggttgtggcaccaagctggaaatcaaagctggagatcaaacgggctgatgctgcaccaacttatctatcttcccaccatccacggaacagttagcaactggaggtgcctcagtcgtgtgcctcatgaacaacttctatcccagagacatcagtgtcaagtggaagattgatggcactgaacgacgagatggtgtcctggacagtgttactgatcaggacagcaaagacagcacgtacagcatgagcagcaccctctcgttgaccaaggctgactatgaaagtcataacctctatacctgtgaggttgttcataagacatcatcctaa

SEQ ID NO: 9 - DNA, 490 bp, Rattus norvegicus
atgaagttcgggttcagcttggttttccttgtccttttcataaaaggtgtccagtgtgaggtgcagctggtggagtctgggggaggcttagtgcagcctggaaggtccctgaaactctcctgtgcagcctcaggattcactttcagtaactatgacatggcctgggtccgccaggctccagatacgaagtgtctggagtgggtcgcatccattagtcctagtggtgatatcacttactatcgagactccgtgaagggccgattcactgtctccagagataatacaaaaagcaccctattcctgcaaatggacagtctgaggtctgaggacacggccacttattactgtgcaagacatgggcctacggaggcccccgatattatggatgcctggggtcaaggagcttcagtcactgtctcctcagctgaaacaacagccccatctgtctatccactggctcctggaactgctctcaaaagtaacatggtga

SEQ ID NO: 10 - DNA, 661 bp, Rattus norvegicus
atggtcctcatatccttgctgttctggatttctgggacattgtgatgacccaatctccatcctctctggctgtgtcagcaggagagacggtcactataaactgcaagtccagtcagagtcttttatacagtggaaaccaaaagaactacttggcctggtaccagcagaaaccagggcagtctcctaaactgctgatccactgggcatctactaggcaatctggtgtccctgatcgcttcataggcagtggatctgggacagacttcactctgaccatcagcagtgtgcaggcagaagatctggcaatttattactgtcagcagtattatgatagtccgctcacgttcggttctgggaccaagctggagatcaaacgggctgatgctgcaccaacttatctatcttcccaccatccacggaacagttagcaactggaggtgcctcagtcgtgtgcctcatgaacaacttctatcccagagacatcagtgtcaagtggaagattgatggcactgaacgacgagatggtgtcctggacagtgttactgatcaggacagcaaagacagcacgtacagcatgagcagcaccctctcgttgaccaaggctgactatgaaagtcataacctctatacctgtgaggttgttcataagacatcatcctaa

SEQ ID NO: 11 - protein, 468 aa, Artificial
  Immunotoxin
EVQLQQSGAELVKPGASVRLSCTASGFNIKDTYMYWVKQRPEQCLEWIGRLDPANGNTRYDPKFQGKATITSDTSSNTAYLQLSSLTSEDTAVYYCAGDYGIIDYWGQGTTLTVSSKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNVPTGAEFLGDGGDVSFSTRGTQNWTVERLLQAHRQLEERVYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 12 - protein, 112 aa, Mus musculus
DIVMSQSPSSLAVSAGEKVTMSCKSSQSLLNSRTRKTYLAWYQQKPGQSPKLLIYWASTRESGVPDRFTGSGSGTEFTLTISSVQAEDLAVYYCKQSYNLWTFGCGTKLEIK

SEQ ID NO: 13 - protein, 473 aa, Artificial
  Immunotoxin
QVQLQQSGAELMKPGASLKISCKATGYTFSSYWIEWVKQRPGHGLQWIGEILPGSGSSNYNEKFKGKATFTADTSSNTAYMQLSSLTSEDSAVYYCARRGSSGSYFAMDYWGQGTSVTVSSKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNVPTGAEFLGDGGDVSFSTRGTQNWTVERLLQAHRQLEERVYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 14 - protein, 110 aa, Artificial
  Mus musculus
DIQMTQSSSSFSVSLGDRVTITCKASEDIYNRLAWYQQKPGNAPRLLISGATSLETGVPLRFSGSGSGKDYTLSITLSLQTEDIATYYCQQYWSTPWTWTFGCGTKLEIK

SEQ ID NO: 15 - DNA, 27 bp, Artificial
  primer
agaaagacatgggtctggaaatggatg

SEQ ID NO: 16 - DNA, 26 bp, Artificial
  primer
tctagaaagacatggcctggaaacag

SEQ ID NO: 17 - DNA, 28 bp, Artificial
  primer
gactgaactcagccaaggagccagagtt

SEQ ID NO: 18 - DNA, 20 bp, Artificial
  primer
cccaacatggatcaggaact

SEQ ID NO: 19 - DNA, 21 bp, Artificial
  primer
caccatggagttacttttgag

SEQ ID NO: 20 - DNA, 35 bp, Artificial
  primer
cagaggcctgaacattgtctggagtggattggaag

SEQ ID NO: 21 - DNA, 35 bp, Artificial
  primer
cttccaatccactccagacactgttcaggcctctg

SEQ ID NO: 22 - DNA, 38 bp, Artificial
  primer
gtccgccaggctccaacgaagtgtctggagtgggtcgc

SEQ ID NO: 23 - DNA, 38 bp, Artificial
  primer
gcgacccactccagacacttcgttggagcctggcggac

SEQ ID NO: 24 - DNA, 41 bp, Artificial
  primer
taagaaggagatatacatatggaggttcagctgcagcagtc

SEQ ID NO: 25 - DNA, 44 bp, Artificial
  primer
gccctcgggacctccggaagcttttgaggagactgtgagagtgg

SEQ ID NO: 26 - DNA, 33 bp, Artificial
  primer
atacatatggaggtgcagctggtggagtctggg

SEQ ID NO: 27 - DNA, 33 bp, Artificial
  primer
tccggaagcttttgaggagacagtgactgaagc

SEQ ID NO: 28 - DNA, 41 bp, Artificial
  primer
taagaaggagatatacatatggacattgtgatgtcacaatc

SEQ ID NO: 29 - DNA, 57 bp, Artificial
  primer
gctttgttagcagccgaattcctatttgatttccagcttggtgccacaaccgaacgt

SEQ ID NO: 30 - DNA, 35 bp, Artificial
  primer
atacatatggacattgtgatgcccaatctccatcc

SEQ ID NO: 31 - DNA, 57 bp, Artificial; identical to SEQ ID NO: 29
  primer